SEQUENCE LISTING (111 sequences)

SEQ ID NO: 1 - DNA, 20 bp, Artificial sequence
  Chemically synthesized primer
tgcatgaagcatttccacat

SEQ ID NO: 2 - DNA, 20 bp, Artificial sequence
  Chemically synthesized primer
tgcagcatctctctccagaa

SEQ ID NO: 3 - DNA, 20 bp, Artificial sequence
  Chemically synthesized primer
tgcatgaagcatttccacat

SEQ ID NO: 4 - DNA, 20 bp, Artificial sequence
  Chemically synthesized primer
tgcagcatctctctccagaa

SEQ ID NO: 5 - DNA, 20 bp, Artificial sequence
  Chemically synthesized primer
gttcctcctccaacaccaag

SEQ ID NO: 6 - DNA, 20 bp, Artificial sequence
  Chemically synthesized primer
ggtgcatcctcactctccac

SEQ ID NO: 7 - DNA, 20 bp, Artificial sequence
  Chemically synthesized primer
gttcctcctccaacaccaag

SEQ ID NO: 8 - DNA, 20 bp, Artificial sequence
  Chemically synthesized primer
ggtgcatcctcactctccac

SEQ ID NO: 9 - DNA, 20 bp, Artificial sequence
  Chemically synthesized primer
ccagatcaatcagggtgaca

SEQ ID NO: 10 - DNA, 21 bp, Artificial sequence
  Chemically synthesized primer
tgactggctcactcgaaatct

SEQ ID NO: 11 - DNA, 20 bp, Artificial sequence
  Chemically synthesized primer
gcaagaaacacaggcaacaa

SEQ ID NO: 12 - DNA, 20 bp, Artificial sequence
  Chemically synthesized primer
ctggctccaaacagcaattc

SEQ ID NO: 13 - DNA, 20 bp, Artificial sequence
  Chemically synthesized primer
ccagatcaatcagggtgaca

SEQ ID NO: 14 - DNA, 21 bp, Artificial sequence
  Chemically synthesized primer
tgactggctcactcgaaatct

SEQ ID NO: 15 - DNA, 20 bp, Artificial sequence
  Chemically synthesized primer
cagctgcactggcactagac

SEQ ID NO: 16 - DNA, 21 bp, Artificial sequence
  Chemically synthesized primer
tctgccttgaatgacagtgtg

SEQ ID NO: 17 - DNA, 20 bp, Artificial sequence
  Chemically synthesized primer
atgcaagctatgctcggact

SEQ ID NO: 18 - DNA, 20 bp, Artificial sequence
  Chemically synthesized primer
ggacccactgtgttctgtga

SEQ ID NO: 19 - DNA, 20 bp, Artificial sequence
  Chemically synthesized primer
atgcaagctatgctcggact

SEQ ID NO: 20 - DNA, 20 bp, Artificial sequence
  Chemically synthesized primer
ggacccactgtgttctgtga

SEQ ID NO: 21 - DNA, 21 bp, Artificial sequence
  Chemically synthesized primer
tggttggattcatgtcaggat

SEQ ID NO: 22 - DNA, 20 bp, Artificial sequence
  Chemically synthesized primer
acttgcccaggagagaatca

SEQ ID NO: 23 - DNA, 21 bp, Artificial sequence
  Chemically synthesized primer
tggttggattcatgtcaggat

SEQ ID NO: 24 - DNA, 20 bp, Artificial sequence
  Chemically synthesized primer
acttgcccaggagagaatca

SEQ ID NO: 25 - DNA, 20 bp, Artificial sequence
  Chemically synthesized primer
tattcgacagcatgccaatc

SEQ ID NO: 26 - DNA, 20 bp, Artificial sequence
  Chemically synthesized primer
actccaaagcctcttgctca

SEQ ID NO: 27 - DNA, 20 bp, Artificial sequence
  Chemically synthesized primer
tattcgacagcatgccaatc

SEQ ID NO: 28 - DNA, 20 bp, Artificial sequence
  Chemically synthesized primer
actccaaagcctcttgctca

SEQ ID NO: 29 - DNA, 20 bp, Artificial sequence
  Chemically synthesized primer
ccaccaaattagcctggaca

SEQ ID NO: 30 - DNA, 20 bp, Artificial sequence
  Chemically synthesized primer
cgcgacccttaggtattctg

SEQ ID NO: 31 - DNA, 20 bp, Artificial sequence
  Chemically synthesized primer
ccaccaaattagcctggaca

SEQ ID NO: 32 - DNA, 20 bp, Artificial sequence
  Chemically synthesized primer
cgcgacccttaggtattctg

SEQ ID NO: 33 - DNA, 20 bp, Artificial sequence
  Chemically synthesized primer
ccaccaaattagcctggaca

SEQ ID NO: 34 - DNA, 20 bp, Artificial sequence
  Chemically synthesized primer
cgcgacccttaggtattctg

SEQ ID NO: 35 - DNA, 20 bp, Artificial sequence
  Chemically synthesized primer
ccagggctggaatactgcta

SEQ ID NO: 36 - DNA, 20 bp, Artificial sequence
  Chemically synthesized primer
cttggaggccagatactcca

SEQ ID NO: 37 - DNA, 20 bp, Artificial sequence
  Chemically synthesized primer
ccagggctggaatactgcta

SEQ ID NO: 38 - DNA, 20 bp, Artificial sequence
  Chemically synthesized primer
cttggaggccagatactcca

SEQ ID NO: 39 - DNA, 20 bp, Artificial sequence
  Chemically synthesized primer
ccagaggaggaacgagctaa

SEQ ID NO: 40 - DNA, 20 bp, Artificial sequence
  Chemically synthesized primer
ttggacggacaggatgtatg

SEQ ID NO: 41 - DNA, 20 bp, Artificial sequence
  Chemically synthesized primer
ccagaggaggaacgagctaa

SEQ ID NO: 42 - DNA, 20 bp, Artificial sequence
  Chemically synthesized primer
ttggacggacaggatgtatg

SEQ ID NO: 43 - DNA, 20 bp, Artificial sequence
  Chemically synthesized primer
ccagaggaggaacgagctaa

SEQ ID NO: 44 - DNA, 20 bp, Artificial sequence
  Chemically synthesized primer
ttggacggacaggatgtatg

SEQ ID NO: 45 - DNA, 20 bp, Artificial sequence
  Chemically synthesized primer
ccagaggaggaacgagctaa

SEQ ID NO: 46 - DNA, 20 bp, Artificial sequence
  Chemically synthesized primer
ttggacggacaggatgtatg

SEQ ID NO: 47 - DNA, 20 bp, Artificial sequence
  Chemically synthesized primer
ccagaggaggaacgagctaa

SEQ ID NO: 48 - DNA, 20 bp, Artificial sequence
  Chemically synthesized primer
ttggacggacaggatgtatg

SEQ ID NO: 49 - DNA, 20 bp, Artificial sequence
  Chemically synthesized primer
gtgcggaggaagaaagtgag

SEQ ID NO: 50 - DNA, 20 bp, Artificial sequence
  Chemically synthesized primer
gaggcagtgattccaagctg

SEQ ID NO: 51 - DNA, 20 bp, Artificial sequence
  Chemically synthesized primer
tctagctgctggcactggta

SEQ ID NO: 52 - DNA, 20 bp, Artificial sequence
  Chemically synthesized primer
gctgaaggaagcctgtgttc

SEQ ID NO: 53 - DNA, 20 bp, Artificial sequence
  Chemically synthesized primer
acgaggtgtgtggtgttcaa

SEQ ID NO: 54 - DNA, 20 bp, Artificial sequence
  Chemically synthesized primer
agttgatgccagctccatgt

SEQ ID NO: 55 - DNA, 20 bp, Artificial sequence
  Chemically synthesized primer
acgaggtgtgtggtgttcaa

SEQ ID NO: 56 - DNA, 20 bp, Artificial sequence
  Chemically synthesized primer
agttgatgccagctccatgt

SEQ ID NO: 57 - DNA, 20 bp, Artificial sequence
  Chemically synthesized primer
gcgctacctgattccaattc

SEQ ID NO: 58 - DNA, 20 bp, Artificial sequence
  Chemically synthesized primer
caacgcaccgaatagttacg

SEQ ID NO: 59 - DNA, 20 bp, Artificial sequence
  Chemically synthesized primer
gcgctacctgattccaattc

SEQ ID NO: 60 - DNA, 20 bp, Artificial sequence
  Chemically synthesized primer
caacgcaccgaatagttacg

SEQ ID NO: 61 - DNA, 20 bp, Artificial sequence
  Chemically synthesized primer
gcgctacctgattccaattc

SEQ ID NO: 62 - DNA, 20 bp, Artificial sequence
  Chemically synthesized primer
caacgcaccgaatagttacg

SEQ ID NO: 63 - DNA, 20 bp, Artificial sequence
  Chemically synthesized primer
gcgctacctgattccaattc

SEQ ID NO: 64 - DNA, 20 bp, Artificial sequence
  Chemically synthesized primer
caacgcaccgaatagttacg

SEQ ID NO: 65 - DNA, 20 bp, Artificial sequence
  Chemically synthesized primer
gcgctacctgattccaattc

SEQ ID NO: 66 - DNA, 20 bp, Artificial sequence
  Chemically synthesized primer
caacgcaccgaatagttacg

SEQ ID NO: 67 - DNA, 20 bp, Artificial sequence
  Chemically synthesized primer
gcgctacctgattccaattc

SEQ ID NO: 68 - DNA, 20 bp, Artificial sequence
  Chemically synthesized primer
caacgcaccgaatagttacg

SEQ ID NO: 69 - DNA, 20 bp, Artificial sequence
  Chemically synthesized primer
ggcagagctgaagtggaaac

SEQ ID NO: 70 - DNA, 21 bp, Artificial sequence
  Chemically synthesized primer
aggtatcagcaagcaattgga

SEQ ID NO: 71 - DNA, 20 bp, Artificial sequence
  Chemically synthesized primer
atgtggcgggactctttatg

SEQ ID NO: 72 - DNA, 20 bp, Artificial sequence
  Chemically synthesized primer
agcggctcaactctcaaact

SEQ ID NO: 73 - DNA, 20 bp, Artificial sequence
  Chemically synthesized primer
atgtggcgggactctttatg

SEQ ID NO: 74 - DNA, 20 bp, Artificial sequence
  Chemically synthesized primer
agcggctcaactctcaaact

SEQ ID NO: 75 - DNA, 20 bp, Artificial sequence
  Chemically synthesized primer
tgggcagagtgaaatcatca

SEQ ID NO: 76 - DNA, 20 bp, Artificial sequence
  Chemically synthesized primer
tagcatgtgttcgcccataa

SEQ ID NO: 77 - DNA, 22 bp, Artificial sequence
  Chemically synthesized primer
tcctgacacacagtcccgagta

SEQ ID NO: 78 - DNA, 20 bp, Artificial sequence
  Chemically synthesized primer
gtttagaaggtcgcgtgcat

SEQ ID NO: 79 - DNA, 22 bp, Artificial sequence
  Chemically synthesized primer
tcctgacacacagtcccgagta

SEQ ID NO: 80 - DNA, 20 bp, Artificial sequence
  Chemically synthesized primer
gtttagaaggtcgcgtgcat

SEQ ID NO: 81 - DNA, 20 bp, Artificial sequence
  Chemically synthesized primer
tctgatgcgccagtttctaa

SEQ ID NO: 82 - DNA, 20 bp, Artificial sequence
  Chemically synthesized primer
ttccaccacttgtcaccaga

SEQ ID NO: 83 - DNA, 20 bp, Artificial sequence
  Chemically synthesized primer
tctgatgcgccagtttctaa

SEQ ID NO: 84 - DNA, 20 bp, Artificial sequence
  Chemically synthesized primer
ttccaccacttgtcaccaga

SEQ ID NO: 85 - DNA, 20 bp, Artificial sequence
  Chemically synthesized primer
aagtggatggttccatttgc

SEQ ID NO: 86 - DNA, 21 bp, Artificial sequence
  Chemically synthesized primer
caaatccaagctgtctctgtg

SEQ ID NO: 87 - DNA, 20 bp, Artificial sequence
  Chemically synthesized primer
aagtggatggttccatttgc

SEQ ID NO: 88 - DNA, 21 bp, Artificial sequence
  Chemically synthesized primer
caaatccaagctgtctctgtg

SEQ ID NO: 89 - DNA, 20 bp, Artificial sequence
  Chemically synthesized primer
cggaaagcatggaaatagga

SEQ ID NO: 90 - DNA, 20 bp, Artificial sequence
  Chemically synthesized primer
aaccggaaactgcaggtaga

SEQ ID NO: 91 - DNA, 20 bp, Artificial sequence
  Chemically synthesized primer
cggaaagcatggaaatagga

SEQ ID NO: 92 - DNA, 20 bp, Artificial sequence
  Chemically synthesized primer
aaccggaaactgcaggtaga

SEQ ID NO: 93 - DNA, 20 bp, Artificial sequence
  Chemically synthesized primer
cggaaagcatggaaatagga

SEQ ID NO: 94 - DNA, 20 bp, Artificial sequence
  Chemically synthesized primer
aaccggaaactgcaggtaga

SEQ ID NO: 95 - DNA, 20 bp, Artificial sequence
  Chemically synthesized primer
cggaaagcatggaaatagga

SEQ ID NO: 96 - DNA, 20 bp, Artificial sequence
  Chemically synthesized primer
aaccggaaactgcaggtaga

SEQ ID NO: 97 - DNA, 20 bp, Artificial sequence
  Chemically synthesized primer
aaagccgctcaactacatgg

SEQ ID NO: 98 - DNA, 21 bp, Artificial sequence
  Chemically synthesized primer
tgctttgaatgcgtcccagag

SEQ ID NO: 99 - DNA, 20 bp, Artificial sequence
  Chemically synthesized primer
aaatctggcaccacaccttc

SEQ ID NO: 100 - DNA, 20 bp, Artificial sequence
  Chemically synthesized primer
cagaggcgtacagggatagc

SEQ ID NO: 101 - DNA, 20 bp, Artificial sequence
  Chemically synthesized primer
aaggctgcaagattcgatgt

SEQ ID NO: 102 - DNA, 20 bp, Artificial sequence
  Chemically synthesized primer
tatcagccggaaagatgagg

SEQ ID NO: 103 - DNA, 20 bp, Artificial sequence
  Chemically synthesized primer
gcaatgcacgtgaagcatac

SEQ ID NO: 104 - DNA, 20 bp, Artificial sequence
  Chemically synthesized primer
gagtggctgggtgttctcat

SEQ ID NO: 105 - DNA, 20 bp, Artificial sequence
  Chemically synthesized primer
cctgtctttgcgaaagcaat

SEQ ID NO: 106 - DNA, 21 bp, Artificial sequence
  Chemically synthesized primer
tgactttcacgatgtcaccaa

SEQ ID NO: 107 - DNA, 20 bp, Artificial sequence
  Chemically synthesized primer
aagaaagcacagctcggaaa

SEQ ID NO: 108 - DNA, 20 bp, Artificial sequence
  Chemically synthesized primer
taggccacatcagacagcag

SEQ ID NO: 109 - DNA, 1849 bp, Homo sapiens
gctaactcaagggagacgtctggtgaacacccgtgggatctaaagaacaagctctgaaagtgttccagctgaaatttcagatcggacagactcgctgcggctccggaggcagtgattccaagctgctcgcgcacgctgctgccaagctgcaggatggtgcacgtagccaggctgctgctgctgctcctcactttcttcctccgcacggatgctgagacacctccaaggtttacacgaacacccgttgatcagacaggggtctctggcggagttgcctctttcatctgccaagctacgggagacccaagacctaaaattgtctggaacaaaaaaggaaagaaagtcagcaatcagagatttgaggtaatagagtttgacgatgggtctggatcagttctcagaatacaacccttacggactccgagggatgaggccatttatgaatgtgtggcctcaaataatgtgggagaaataagtgtatccaccagactcacagttttgcgggaagatcaaattcccaggggcttccctaccattgacatgggcccacagttgaaggtggttgagcgtactcgcacggccaccatgctttgtgcagccagtggtaatccggatccagaaatcacttggtttaaagatttcttacctgtggacacaagcaacaacaatggtcgtattaagcagttacgatcaggagcccttcagattgagcagagtgaagagtctgaccaaggaaaatatgagtgtgttgccaccaacagcgcgggcactcgctattccgctcctgccaatttatatgtcagagttcgccgtgtcccaccaagattctctatcccacccactaatcatgaaatcatgccaggcggaagcgttaatatcacctgtgtggccgtggggtcaccaatgccttatgtaaagtggatgttgggggcagaagatctgacacctgaagatgatatgccaataggaagaaatgtgctagaactgaatgatgtaagacagtcagcaaattacacctgtgttgctatgtcaacactgggtgtcattgaagcaatagcacagatcactgtcaaagccttacccaaacctccaggaactcctgtagtgaccgagagcacagctacaagcatcacactgacgtgggactctgggaaccctgagcctgtttcttattacataattcagcataaacctaaaaactctgaggaactttacaaagaaattgatggggtggcgaccacacgctacagtgtcgctggactaagtccctactcggattatgaattcagggttgttgctgtcaataacattgggcgggggcctcccagcgaacctgtgctaacacaaacctcagagcaagcaccatccagtgccccgagggatgtccaggcacgaatgttgagttcgaccaccattttggtacagtggaaggaacctgaagagccaaatggacagatccaaggatatagagtttattatacaatggatcccactcaacatgtcaacaactggatgaaacacaatgtagctgacagccaaatcactactattggcaacttagtgccccagaaaacatattctgtcaaagtcctggcttttacctcaattggagatggtcccctttcaagtgacatacaagtcatcactcagacaggagtaccagggcagccactaaacttcaaagcagaacctgagtctgaaacaagtattttgctctcttggacacctccacgttcagataccattgccaactatgaactggtctacaaagatggggagcatggagaggagcaacgaattaccattgagccagggacatcatataggctgcaaggactgaaaccaaaca

SEQ ID NO: 110 - DNA, 1815 bp, Homo sapiens
cggcgccgccagccagcctcggcggggagagtgcagtttattctgggcctgtgtggaatagaaagacaattctttatttgactggtaggaaagaaaagagaagactcctcagggtttccaaggaggactggagcggctgctttagtgaagaagtgaagaatcaaggaagtaaaggacctgtctcaaagtcacacaactaggaaatcttgaggagtttgatacttttcatcctccagctataaaatcatcttgtagggggaagctgtacattggtggaaaataaaaggcaagcagttcctggcccgtgatattccagttagaggtgtctgactgacagcatgttggcaccgagggaagcgatgggcttcggctccaggtttctgtcctgagaccacagcagaatgtgccttggacaaaggcttggactccttgatcttctggtaacttcaaaaatctggtctcagggtggtgaactttttcttcgaattcctaacctgtgagcttacgaaaaagggagcatgcctgtcttattctcagtgcctcatgggaaataaaagattgggagatgatagctacctgatgatggtgcctgtccattaactgaacaactgtggttttgaacttcttgaggccagggactgtgtctgaaaacaccgacggtggctaactcaagggagacgtctggtgaacacccgtgggatctaaagaacaagctctgaaagtgttccagctgaaatttcagatcggacagactcgctgcggctccggaggcagtgattccaagctgctcgcgcacgctgctgccaagctgcaggatggtgcacgtagccaggctgctgctgctgctcctcactttcttcctccgcacggatgctgagacacctccaaggtttacacgaacacccgttgatcagacaggggtctctggcggagttgcctctttcatctgccaagctacgggagacccaagacctaaaattgtctggaacaaaaaaggaaagaaagtcagcaatcagagatttgaggtaatagagtttgacgatgggtctggatcagttctcagaatacaacccttacggactccgagggatgaggccatttatgaatgtgtggcctcaaataatgtgggagaaataagtgtatccaccagactcacagttttgcgggaagatcaaattcccaggggcttccctaccattgacatgggcccacagttgaaggtggttgagcgtactcgcacggccaccatgctttgtgcagccagtggtaatccggatccagaaatcacttggtttaaagatttcttacctgtggacacaagcaacaacaatggtcgtattaagcagttacgatcaggtactgatttttctccctcttctctttctctgttacttaatgggtggtccatgtttgtgatgtcatagcctgtttcagagtctgtctttctccttttctctgtgtgttttgcagcttctgtttaatccacattgctcactgctgtgactgtatttttggtaattttttaatttactgctttttcgcagtatttgatggatccattttctcctctttctttcttctttctctgttttcttttgaaacaaatttatttcaacattggacttcattcagaacttgcaaggagatccaatggttgtatccattaaaaaataaaatgttcatattaaaccctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggggggggcc

SEQ ID NO: 111 - DNA, 2475 bp, Mus musculus
ggccccgccccgccccgcccgcgcgcaccgacgcacctgcgggcactcgccccggcgtctgcgggacccgcgggctccgagcgcgccgccaccctcccgtgccgacctcgcacacgcagataccacttttaaagaagccgaagcgctgcagcccaccgacgccgagcgagcgctgccggctgaggccgttgctctccctgtctgcagttgagcccggaccctgggacagaaagacagttctcggttggactggaagaaagggaaggaagactcctctgttcccgaggagagcggagcggctgccctagtgaagaaatgaagaatcaaagaagtaaaggacttatctcataatcacataactagggaaccttgaggagttgcacactttccatccccagtcactgctccccttcggggggagggtcttacttgctcgaaatagaaggcaagcagttcttggcctgtgaaatctgagtggcagtgcctggctgtcagtatgctggccccaatgttgatggacatgattagctttggctccaggttcctgtcctgagaccacagcagaaagtgccttggatgaaggcttggactctggatcgtctggtgacttcaactgtctggtctcagggtggagaactttctttgtatgcctaacctgtgagtcgatgatgatggtgcctgtccactaactgaacaactgtggtgttcaacattctgaggccaggaactgtttctgaaacactctacagagctcatagatgggagaagtctggtgagcacccaggggatctaactagcaagctctaaaagggttccagctgaagtttctgagcggagcagtcgctgtggctccctaggctgagtttccaagctgctggttcatgccgttgacaaactgcaggatggtgcccgtggccaggccgctgtcgctgctcctcaccttcttcctctgcgcctgtgctgagacaccccccaggtttacacgaactccggttgatcagacaggggtctctggaggagttgcatcattcatttgtcaagctacaggagacccaagacctaaaattgtctggaacaaaaaaggaaagaaagtcagcaaccagagatttgaggtaatagaatttgacgatggatctggatcagtactcagaatacagcccttaaggactccacgggatgaggccatttatgaatgtgtggcctcgaataatgtgggagaaatcagtgtgtccacaagactcacagttttacgtgaggatcagattcctagagggttccctacgattgatatgggcccgcagttgaaggtggtggaacggacccgcaccgccaccatgctgtgtgcagccagcggtaatccggatccagaaatcacttggtttaaagatttcttacctgttgacacaagcaacaacaatgatcgtattaagcagttacgatcaggtattgatttatcttcctcttctctttctctgttatttaatatgttgtccatgtttgtgaggccatggcctcatccagaacctgtcttcccttggtgtgttttgcggcttctgtttaatccacattgctcactgctgtgactgcattcttaataatttttaatttactgctttttctgcggtatttgatggatccattttctcttctttcttctttcttcgttttcttttgaaacaaatttatttcaacattgaatttcattccaaacttgcaaagcgctccagtggctatttccatatgaaaatgttcatattaaacctgaaaataattcagttgtactcaagtgtattttctgctttcttttctacatttaaactcatccaggtagtaattgtcagtgagtttgggcacatctgctttcttttcactaatacacaatacacagaaacgtggattcctgagtgaccagatgtatttttttaaactccttttcgaagtaccaatgaaagtgagggatttgattaactccatttctgtgtcattgttagtgagcttggcccacacagtagctgtggcaatatatgtgcccatgctcgagggctttgacccctccagtgactggatctacagaagtctgctttggtgtagactccagctttggccgttttctttctctttactccaactccgtgtcccgttctcatcccaggttattcctttgctctctatagattaaaatagaaaacattctaaatttcttagagcctgtccctaggcaatttcacagaaatgtatactaataatctctgtgctgtgactttggtaataatattgatagaaaattctaagttgtgaactgcctgtcttcccagaattctcagcttttgccctgtcctgtgatggtaaagcgtcttgttaactggctcttttgcttgttgtcagcagaactgtgtagattagcattctgttgttgtaaataaataattattcacatgtaacatagatgatttagtataataaagctttaactcc

(19) United States
(12) Patent Application Publication
(10) Pub. No.: US 2019/0156913 A1
(43) Pub. Date: May 23, 2019

(54) METHODS OF DIAGNOSING AND TREATING CANCER BY DETECTION OF CHROMOSOMAL ABNORMALITIES

(71) Applicant: Dana-Farber Cancer Institute, Inc., Boston, MA (US)

(72) Inventors: Matthew MEYERSON, Concord, MA (US); Barbara WEIR, Brookline, MA (US); Xiaojun ZHAO, Quincy, MA (US)

(21) Appl. No.: 16/019,079

(22) Filed: Jun. 26, 2018

Related U.S. Application Data
(63) Continuation of application No. 11/921,098, filed on Dec. 21, 2009, now abandoned.
(60) Provisional application No. 60/685,978, filed on May 31, 2005.
(60) Provisional application No. 60/685,635, filed on May 27, 2005.

(51) Int. Cl. G16B 20/20; C12Q 1/6837; C12Q 1/6886; G16H 50/20; G16B 20/10; G16H 50/30

(52) U.S. Cl. CPC G16B 20/20; C12Q 1/6837; C12Q 1/6886; G16H 50/20; C12Q 2600/158; G16H 50/30; C12Q 2600/112; C12Q 2600/118; C12Q 2600/156; G16B 20/10

(57) ABSTRACT

High-density arrays, representing approximately 115,000 single nucleotide polymorphism (SNP) loci, were used to measure genome-wide copy number changes in primary human lung carcinoma specimens and cell lines derived from human lung carcinomas. Changes in DNA copy number contribute to cancer pathogenesis. Recurrent high-level amplifications and homozygous deletions were identified. Systematic copy number analysis identified high-level amplification of numerous genetic loci.

### RELATED APPLICATIONS

This application is a continuation of and claims priority to U.S. Ser. No. 11/921,098, filed on Dec. 21, 2009, which is a national stage application, filed under 35 U.S.C. § 371, of International Application No. PCT/US2006/021078, filed on May 30, 2006, which claims the benefit of U.S. Ser. No. 60/685,635, filed May 27, 2005 and 60/685,978, filed May 31, 2005, each of which is incorporated herein by reference in its entirety.

### GOVERNMENT INTEREST

This invention was supported in part by National Institutes of Health grant 2P30 CA06516-39 and National Cancer Institute grants R01CA92824, P50CA70907, 2P30 CA06516-39, 1K12CA87723-01 and CA58207. The United States government may have certain rights in the invention.

### FIELD OF THE INVENTION

This invention relates to method of diagnosing cancer.

### BACKGROUND OF THE INVENTION

Cancer occurs through the accumulation of genetic defects, including the hyperactivation of oncogenes, which normally stimulate cell growth, and the inactivation of tumor suppressor genes (TSGs), which normally repress cell growth. These changes can occur through somatic point mutations, small deletions, or other large chromosomal copy number aberrations such as amplification, deletion or loss of heterozygosity (LOH).

The mapping of copy number alterations and regions of LOH have successfully revealed regions important for tumorigenesis and have resulted in the subsequent identification of likely tumor suppressor genes and oncogenes.

Lung cancer is the leading cause of cancer deaths in the world and is estimated to result in approximately 150,000 deaths annually in the United States alone. Lung cancer is categorized into two major groups based on histopathologic features, small cell carcinoma (SCLC) and non-small cell carcinoma (NSCLC). The major subtypes of NSCLC are squamous cell carcinoma, adenocarcinoma, and large cell carcinoma (LC).

The development and progression of lung cancer is a multi-step and complex process, resulting in the accumulation of a series of genetic defects during tumorigenesis (Sekido et al., Annu Rev Med, 54: 73-87, 2003). To date, consistent regions of chromosomal aberrations in lung cancer have been identified by cytogenetic techniques, comparative genomic hybridization (CGH) and LOH analyses (Sy et al., Eur J Cancer, 40: 1082-1094, 2004; Testa et al. Cancer Genet Cytogenet, 95: 20-32, 1997; Balsara et al., Oncogene, 21: 6877-6883, 2002; Stanton et al., Genes Chromosomes Cancer, 27: 323-331, 2000). In SCLC the most frequent gains have been localized to 3q, 5p, 8q, and frequent regions of loss include 3p, 4q, 5q, 8p, 9p, 10q, 13q, 17p (Balsara et al., 2002) as well as 15q and 16q (Stanton at al., Genes Chromosomes Cancer, 27: 323-331, 2000). The most frequent regions of gain in NSCLC are 1q, 3q, 5p, 8q, and frequent losses occur in 3p, 8p, 9p, 13q, and 17p (Balsara et al., 2002). Those regions often encompass oncogenes such as PIK3CA on 3q and MYC on 8q, and tumor suppressor genes such as CDKN2A on 9p, PTEN on 10q, RB1 on 13q, and p53 on 17p.

Amplifications in NSCLC often target receptor tyrosine kinase (RTK) genes including the epidermal growth factor receptor gene, EGFR (Reissmann et al., J Cancer Res Clin Oncol, 125: 61-70, 1999). RTKs have become attractive drug targets in cancer given the effectiveness of RTK inhibitors such as imatinib, trastuzamab, erlotinib, and gefitinib. Thus genome-wide studies of RTKs are potentially of great clinical significance, as found with the identification of somatic mutations in the EGFR gene of lung cancer patients. The compound gefitinib (IRESSA™), an EGFR kinase inhibitor, has shown activity in the treatment of lung adenocarcinoma patients, primarily in patients from Japan, non-smokers, and women (Miller et al., J Clin Oncol, 22: 1103-1109, 2004). Mutations in the EGFR kinase domain in non-small cell carcinoma specimens were found to correlate closely with patient responses to gefitinib (Paez et al., Science, 304: 1497-1500, 2004; Lynch at al., N Engl J Med, 350: 2129-2139, 2004; Pao et al., Proc Natl Acad Sci USA, 101: 13306-13311, 2004) as well as erlotinib (TARCEVA) (Pao at al., Proc Natl Acad Sci USA, 101: 13306-13311, 2004). The relationship between EGFR amplification and EGFR mutation in lung carcinoma has not been described.

A need exists for better predictors of responses to treatment of neoplasms, including, e.g., lung cancers such as NSCLC and SCLC, and other types of cancer.

### SUMMARY OF THE INVENTION

The invention is based the identification of homozygous deletions and chromosome amplifications across lung cancer genomes.

Accordingly, the invention provides a method of diagnosing cancer or a predisposition thereto in a subject, by determining in a biological sample the copy number of a nucleic acid, e.g., a gene, a chromosome or fragment thereof that is amplified in a cancerous state compared to a non-cancerous state. Detection of a copy number greater than two of the nucleic acid indicates that the subject has cancer or a predisposition to cancer. A symptom of cancer is reduced or alleviated by identifying a subject having an elevated copy number of a nucleic acid compared to a normal non-cancer copy number of the nucleic acid and administering to the subject a compound which inhibits expression or activity the nucleic acid or polypeptide encoded by the nucleic acid. Optionally, the compound inhibits ß-hydroxylase activity.

Nucleic acids include an ASPH gene, a region of human chromosome 8q12.1-q13.11; a MGC24646 gene; a region of human chromosome 12p11; a LOC283343 gene; a CGI-04 gene; a DNM1L gene; a PKP2 gene; a region of human chromosome 22q11; a CRKL gene or a PIK4CA gene.

By copy number is meant the number of copies of a given gene present in a cell or nucleus. A normal somatic cell is a diploid cell, having two copies of each gene chromosome, thus a copy number greater than two indicates an amplification of the gene or chromosome. Copy number is determined by methods known in the art. For example copy number is determined by real time polymerase chain reaction, single nucleotide polymorphism (SNP) arrays, or interphase fluorescent in situ hybridization (FISH) analysis. The nucleic acid is present at a copy number of 4, 5, 10, 15, 20, 25, 30, 35, 40, or more. By region of a human chromosome is meant that fragment of the chromosome is identified. For example, the region is 50, 100, 200, 300, 400, 500, 1000 or more kilobases in length.

In another aspect, the invention provides a method of diagnosing cancer or a predisposition thereto in a subject, by determining in a biological sample the presence of a one or more deletions on chromosome 9p23; a PTPRD tyrosine phosphatase gene; a bc028038 gene; chromosome 3q25; a AADAC gone; or a SUCNR1 gene.

The cancer is lung cancer such as small cell lung cancer, lung adenocarcinoma or large cell carcinoma.

The biological sample is any bodily tissue or fluid that contains DNA. The subject is preferably a mammal. The mammal can be, e.g., a human, non-human primate, mouse, rat, dog, cat, horse, or cow.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIGS. 1A-1D are graphic representations of the measurement of X FIG. 1 is a series of scatter plots showing DNA copy number alterations by SNP array analysis. (A) Scatter plot of log 2 copy number ratios (black dots, left axis) and inferred copy number derived by hidden Markov model (red dots, right axis) versus genomic position of all SNPs, for the NSCLC cell line H2122. The ratios are displayed as a median of each SNP and its two nearest neighbors. High-level amplification within chromosome arm 8q (inferred copy number >=7) and homozygous deletions within chromosome arms 2q, 8q, and 9p (inferred copy number=0) are indicated with red arrows. (B,C) The fraction of samples with copy number amplification of at least 3 copies (red), and copy number reduction to <1.5 copies (blue) across all autosomal SNPs is shown for (B) all NSCLC samples and (C) all SCLC samples. SNP markers are ordered according to their mapped positions from chromosome 1 to 22 with vertical solid and dashed lines (grey) indicating chromosome boundaries and centromeres, respectively. (D) The recurrence of high-level amplification and homozygous deletions across all autosomal SNPs. The number of samples with amplifications of at least copy number 7 is shown with red bars; the number of samples with homozygous deletions is shown with blue bars. The recurrent regions (>=2) that harbor known or candidate oncogene (red) and tumor suppressor genes (blue) are highlighted.

FIGS. 2A and 2B are scatter plots showing homozygous deletions within chromosome 9q23. Scatter plots of log 2 copy number ratios (black dots, left axis) and inferred copy number (red dots, right axis) against the position of all autosomal SNPs show homozygous deletions at chromosome 9p23 in (A) the BA C cell line H358 and (B) the SCLC primary tumor sample S0177T.

FIG. 2C is a illustration showing the positions of the four homozygous deletions (S0177T, H358, HCC1771 and H2347) along chromosome 9 (Mb). The positions of the PTPRD gene and BC028038 spliced transcript are shown in relation to the deletions.

FIG. 3A is a photograph showing interphase FISH of SCLC cell line H2171 with a probe to ASPH (green) and the centromere of chromosome 8 (red).

FIG. 3B is a scatter plot showing 8q12-13 amplification the SCLC sample S0177T.

FIG. 3C is a bar chart showing Real time quantitative PCR copy numbers (blue columns) and inferred copy numbers (violet columns) of a panel of SCLC primary tumors and cell lines.

FIG. 3D is a an illustration showing inferred copy number (Y-axis) of the 8q 12-13 amplicon in sample S0177T (red line) and H2171 (green line) are plotted against position (Mb, X-axis) along chromosome 8. The positions of genes (ASPH and MGC34346) within the region are shown in relation to the amplicons.

FIG. 4A-D are a series of scatter plots of log 2 copy number ratios (black dots, left axis) and inferred copy number (red dots, right axis). (A) Over S-fold FGFR1 amplification in the primary adenocarcinoma sample MOH1 622T on chromosome 8. (B) 4-fold ERBB2 amplification in the adenocarcinoma cell line H1819 on chromosome 17. (C) 5-fold MET amplification in the adenocarcinoma cell line H1993 on chromosome 7. (D) High-level EGFR amplification in the adenocarcinoma cell line HCC827, containing an exon 19 deletion (E746-A750del) in EGFR

FIG. 5 is an illustration showing a global view of copy number alterations across chromosomes 1-X in 101 tumor and cell line samples and 12 normal samples is shown using log 2 signal intensity ratio for each sample. Colors vary from dark blue to dark red representing the range of log 2 ratio. Each column represents a different cell line or tumor, and each row represents SNP markers, ordered by genomic position from p arm (top) to q arm (bottom).

FIG. 6A-D is a series of scatter plots. (A) Deletions of the 3q25 region are shown by scatter plots of log 2 copy number ratios (black dots, left axis) and inferred copy number (red dots, right axis) against chromosomal SNP position using the hg16 genome assembly in non-small cell carcinoma line H2882 and primary small cell carcinoma S0177T. (B) High-level amplifications of the 12p 11 locus are shown by scatter plots of log 2 copy number ratios (black dots, left axis) and inferred copy number (red dots, right axis) against chromosomal SNP position using the hg16 genome assembly in the adenocarcinoma cell line H2087 and primary squamous cell carcinoma S0515T. (C) High-level amplification of the 22q11 locus. Amplifications within the 22q 11 region are shown by scatter plots of log 2 copy number ratios (black dots, left axis) and inferred copy number (red dots, right axis) against chromosomal SNP position using the hg16 genome assembly in adenocarcinoma cell line HCC515 and primary adenocarcinoma S0380T. (D) CDK4 amplification. Scatter plots of log 2 copy number ratios (black dots, left axis) and inferred copy number (red dots, right axis) against the position of all autosomal SNPs show over 4-fold amplification of CDK4 in the adenocarcinoma cell line H2087.

FIG. 7A-C is an illustration showing the alignment of human PTPRD (NM_13091) with PTPRD-like transcripts in human (BC028038) and mouse (AK034145). Nucleotide sequences were aligned with AlignX, a component of the VectorNTI Suite 9.0.0 and exported in MSF format. The alignment file was processed with the GeneDoc program. Shading levels represent 100% (darkest), >80%, >60% and <60% conservation of amino acid residues among the sequences for a given position. Only the first 1849 bp of PTPRD are shown.

### DETAILED DESCRIPTION OF THE INVENTION

The invention is based on the discovery that copy number changes of chromosomal loci for various genes are correlated to a cancerous state. Specifically, multiple homozygous deletions and chromosome amplifications were identified across lung cancer genomes. The study represents the first application of genome-wide copy number analysis in lung cancer by SNP array. This technology provides a unique opportunity to assess DNA copy number changes and LOH simultaneously throughout the entire genome.

To discover novel genomic changes in human lung carcinomas SNP arrays covering approximately 115,000 single nucleotide polymorphism (SNP) loci were used to identify copy number changes in a panel of DNA from 77 NSCLC and 24 SCLC lung cell lines and primary tumors. The high resolution of the SNP arrays, allowed for the identification of several small homozygous deletions and amplifications that have not been detected by previous methods.

In addition to previously characterized loci, two regions of homozygous deletion were found, one near the PTPRD locus on chromosome segment 9p23 in four samples representing both small cell lung carcinoma (SCLC) and non-small cell lung carcinoma (NSCLC) and the second on chromosome segment 3q25 in one sample each of NSCLC and SCLC. High-level amplifications were identified within chromosome segment 8q1213 in two SCLC specimens, 12p11 in two NSCLC specimens, and 22q11 in four NSCLC specimens. Systematic copy number analysis of tyrosine kinase genes identified high-level amplification of EGFR in three NSCLC specimens, FGFR1 in two specimens, and ERBB2 and MET in one specimen each. EGFR amplification was shown to be independent of kinase domain mutational status.

Chromosomal copy number alterations can lead to activation of oncogenes and inactivation of tumor suppressor genes (TSGs) in human cancers. These genes play key roles in multiple genetic pathways to positively and negatively regulate cell growth, proliferation, apoptosis, and metastasis. Many TSGs, including RB1, and PTEN were originally identified by localizing regions of homozygous deletions. Similarly, regions of chromosome amplification frequently harbor oncogenes, such as MYC, and ERBB. Thus, identification of cancer-specific copy number alterations will not only provide new insight into understanding the molecular basis of tumorigenesis but will also facilitate the discovery of new TSGs and oncogenes.

Accordingly, the invention provides diagnostic and prognostic methods for identifying a subject with cancer by identifying one or more amplifications or deletions described herein. Also included in the invention are methods for treating, e.g., alleviating one or more symptoms of cancer by administering to a subject a compound that decreases the expression or activity of an amplified gene, e.g., an ASPH gene, a region of human chromosome 8q12.1-q13.11; a MGC24646 gene; a region of human chromosome 12p11; a LOC283343 gene; a CGI-04 gene; a DNM1L gene; a PKP2 gene; a region of human chromosome 22q11; a CRKL gene or a PIK4CA gene. Alternatively, the subject is administered a compound that increases the expression or activity of deleted gene, e.g., chromosome 9p23; a PTPRD tyrosine phosphatase gene; a bc028038 gene; chromosome 3q25; a AADAC gene; or a SUCNR1 gene.

Diagnostic and Prognostic Methods

The invention provides diagnostic and prognostic methods for identifying a subject with cancer. Cancer is diagnosed by detecting an alteration of copy number of a cancer-associated nucleic acid. The nucleic acids whose copy numbers are modulated (i.e., increased or decreased) in cancer patients are summarized in Table A and are collectively referred to herein as “cancer-associated genes” or “cancer-associated nucleic acids.”

TABLE A
Cancer Associated Nucleic Acids

| Nucleic Acid | Modulation |
|---|---|
| ASPH gene | amplified |
| chromosome 8q12.1-q13.11 | amplified |
| MGC24646 gene | amplified |
| chromosome 12pl1 | amplified |
| LOC283343 gene | amplified |
| CGI-04 gene | amplified |
| DNM1L gene | amplified |
| PKP2 gene | amplified |
| chromosome 22q11 | amplified |
| CRKL gene | amplified |
| PIK4CA gene | amplified |
| chromosome 9p23 | deleted |
| PTPRD tyrosine phosphatase | deleted |
| gene | |
| chromosome 3q25 | deleted |
| bc028038 gene | deleted |
| AADAC gene | deleted |
| SUCNR1 gene | deleted |

Detection of a copy number greater that two in a subject-derived sample of an amplified (i.e., overexpressed) cancer-associated nucleic acid indicates the subject has or is predisposed to developing cancer. Detection of a copy number less than two in a subject-derived sample of a deleted (i.e., underexpressed) cancer associated nucleic acid indicates the subject is predisposed to developing cancer.

Also provided is a method of assessing the prognosis of a subject cancer by comparing the copy number of one or more cancer-associated nucleic acids in a test sample to the copy number in a reference sample from patients over a spectrum of disease stages. By comparing copy number of one or more cancer-associated nucleic acids and the reference sample(s), or by comparing the pattern of gene expression over time in sample derived from the subject, the prognosis of the subject can be assessed.

A decrease in copy number of one or more deleted cancer-associated nucleic acids compared to a normal control or an increase in copy number of one or more of the amplified cancer associated nucleic acids compared a normal control indicates less favorable prognosis. An increase in copy number of one or more deleted cancer-associated nucleic acids indicates a more favorable prognosis, and a decrease in copy number of one or more of the amplified cancer associated nucleic acids indicates a more favorable prognosis for the subject.

Optionally, detection of an amplified cancer-associated gene is determined at the RNA level by detecting an increased amount of the RNA transcript, or at the protein level by detecting an increased amount of the protein encoded by the cancer associated gene compared to a normal control level. Similarly detection of a deleted cancer associated gene is determined by detecting a decreased amount of the RNA transcript or protein encoded by the cancer associated gene compared to a normal control level.

The cancer is lung, upper airway primary or secondary, head or neck, bladder, kidney, pancreas, mouth, throat, pharynx, larynx, esophagus, brain, liver, spleen, lymph node, small intestine, blood cells, colon, stomach, breast, endometrium, prostate, testicle, ovary, skin, bone marrow, muscle, nerve or blood cancer.

The biological sample can be any tissue or fluid that contains nucleic acids. Various embodiments include paraffin imbedded tissue, frozen tissue, surgical fine needle aspirations, cells of the skin, muscle, lung, head and neck, esophagus, kidney, pancreas, mouth, throat, pharynx, larynx, esophagus, facia, brain, prostate, breast, endometrium, small intestine, blood cells, liver, testes, ovaries, uterus, cervix, colon, stomach, spleen, lymph node, bone marrow or kidney. Other embodiments include fluid samples such as bronchial brushes, bronchial washes, bronchial ravages, peripheral blood lymphocytes, lymph fluid, ascites, serous fluid, pleural effusion, sputum, cerebrospinal fluid, lacrimal fluid, esophageal washes, and stool or urinary specimens such as bladder washing and urine.

Methods of evaluating the copy number of a particular gene or chromosomal region are well known to those of skill in the art and include Hybridization-based Assays and Amplification-based Assays.

Hybridization-Based Assays

Hybridization-based assays include, but are not limited to, traditional “direct probe” methods such as Southern Blots or In Situ Hybridization (e.g., FISH), and “comparative probe” methods such as Comparative Genomic Hybridization (COH). The methods can be used in a wide variety of formats including, but not limited to substrate—(e.g. membrane or glass) bound methods or array-based approaches as described below.

In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. The preferred size range is from about 200 bp to about 1000 bases, more preferably between about 400 to about 800 bp for double stranded, nick translated nucleic acids.

In some applications it is necessary to block the hybridization capacity of repetitive sequences. Thus, human genomic DNA or Cot-1 DNA is used to block non-specific hybridization.

In Comparative Genomic Hybridization methods a first collection of (sample) nucleic acids (e.g. from a possible tumor) is labeled with a first label, while a second collection of (control) nucleic acids (e.g. from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number.

Other Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc.

The methods of this invention are particularly well suited to array-based hybridization formats. Arrays are a multiplicity of different “probe” or “target” nucleic acids (or other compounds) attached to one or more surfaces (e.g., solid, membrane, or gel). The multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958.

Arrays, particularly nucleic acid arrays, can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated systems that taps a microcapillary against a surface to deposit a small volume of a biological sample. The process is repeated to generate high density arrays. Arrays can also be produced using oligonucleotide synthesis technology. Thus, for example, U.S. Pat. No. 5,143,854 and PCT patent publication Nos. WO 90/15070 and 92/10092 teach the use of light-directed combinatorial synthesis of high density oligonucleotide arrays.

A spotted array can include genomic DNA, e.g. overlapping clones that provide a high resolution scan of the amplicon corresponding to the region of interest. Amplicon nucleic acid can be obtained from, e.g., MACs, YACs, BACs, PACs, PIs, cosmids, plasmids, inter-Alu PCR products of genomic clones, restriction digests of genomic clone, cDNA clones, amplification (e.g., PCR) products, and the like.

The array nucleic acids are derived from previously mapped libraries of clones spanning or including the target sequences of the invention, as well as clones from other areas of the genome, as described below. The arrays can be hybridized with a single population of sample nucleic acid or can be used with two differentially labeled collections (as with an test sample and a reference sample).

Many methods for immobilizing nucleic acids on a variety of solid surfaces are known in the art A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials which may be employed include paper, ceramics, metals, metalloids, semiconductive materials, cermets or the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g. Denhardt's solution) can be employed to avoid non-specific binding, simplify covalent conjugation, enhance signal detection or the like. If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

For example, methods for immobilizing nucleic acids by introduction of various functional groups to the molecules is known (see, e.g., Bischoff (1987) Anal. Biochem., 164: 336-344; Kremsky (1987) Nucl. Acids Res. 15: 2891-2910). Modified nucleotides can be placed on the target using PCR primers containing the modified nucleotide, or by enzymatic end labeling with modified nucleotides. Use of glass or membrane supports (e.g., nitrocellulose, nylon, polypropylene) for the nucleic acid arrays of the invention is advantageous because of well developed technology employing manual and robotic methods of arraying targets at relatively high element densities. Such membranes are generally available and protocols and equipment for hybridization to membranes is well known.

Target elements of various sizes, ranging from 1 mm diameter down to 1 micron can be used. Smaller target elements containing low amounts of concentrated, fixed probe DNA are used for high complexity comparative hybridizations since the total amount of sample available for binding to each target element will be limited. Thus it is advantageous to have small array target elements that contain a small amount of concentrated probe DNA so that the signal that is obtained is highly localized and bright. Such small array target elements are typically used in arrays with densities greater than 10⁴/cm². Relatively simple approaches capable of quantitative fluorescent imaging of 1 cm² areas have been described that permit acquisition of data from a large number of target elements in a single image (see, e.g., Wittrup (1994) Cytometry 16:206-213).

Arrays on solid surface substrates with much lower fluorescence than membranes, such as glass, quartz, or small beads, can achieve much better sensitivity. Substrates such as glass or fused silica are advantageous in that they provide a very low fluorescence substrate, and a highly efficient hybridization environment. Covalent attachment of the target nucleic acids to glass or synthetic fused silica can be accomplished according to a number of known techniques (described above). Nucleic acids can be conveniently coupled to glass using commercially available reagents. For instance, materials for preparation of silanized glass with a number of functional groups are commercially available or can be prepared using standard techniques (see, e.g., Gait (1984) Oligonucleotide Synthesis: A Practical Approach, IRL Press, Wash., D.C.). Quartz cover slips, which have at least 10-fold lower autofluorescence than glass, can also be silanized.

Alternatively, probes can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith (1992) Science 258: 1122-1126). Biotin or digoxigenin end-labeled nucleic acids can be prepared according to standard techniques. Hybridization to nucleic acids attached to beads is accomplished by suspending them in the hybridization mix, and then depositing them on the glass substrate for analysis after washing. Alternatively, paramagnetic particles, such as ferric oxide particles, with or without avidin coating, can be used.

For example, a probe nucleic acid is spotted onto a surface (e.g., a glass or quartz surface). The nucleic acid is dissolved in a mixture of dimethylsulfoxide (DMSO) and nitrocellulose and spotted onto amino-silane coated glass slides. Small capillary tubes can be used to “spot” the probe mixture.

A variety of other nucleic acid hybridization formats are known to those skilled in the art. For example, common formats include sandwich assays and competition or displacement assays. Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Detection of a hybridization complex may require the binding of a signal generating complex to a duplex of target and probe polynucleotides or nucleic acids. Typically, such binding occurs through ligand and anti-ligand interactions, such as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE-T at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, the wash is performed at the highest stringency that produces consistent results and that provides a signal intensity greater than approximately 10% of the background intensity. The hybridized array may be washed at successively higher stringency solutions and read between each wash. Analysis of the data sets thus produced will reveal a wash stringency above which the hybridization pattern is not appreciably altered and which provides adequate signal for the particular probes of interest.

Background signal is reduced by the use of a detergent (e.g., C-TAB) or a blocking reagent (e.g., sperm DNA, cot-1 DNA, etc.) during the hybridization to reduce non-specific binding. In a particularly preferred embodiment, the hybridization is performed in the presence of about 0.1 to about 0.5 mg/ml DNA (e.g., cot-1 DNA). The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background membranes can be used (see, e.g., Chu (1992) Electrophoresis 13:105-114). The sensitivity for detection of spots (“target elements”) of various diameters on the candidate membranes can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., membranes, glass, fused silica) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

The hybridized nucleic acids are detected by detecting one or more labels attached to the sample or probe nucleic acids. The labels may be incorporated by any of a number of means well known to those of skill in the art. Means of attaching labels to nucleic acids include, for example nick translation or endlabeling (e.g. with a labeled RNA) by kinasing of the nucleic acid and subsequent attachment (ligation) of a nucleic acid linker joining the sample nucleic acid to a label (e.g., a fluorophore). A wide variety of linkers for the attachment of labels to nucleic acids are also known. In addition, intercalating dyes and fluorescent nucleotides can also be used.

Detectable labels suitable for use in the present invention include any composition detectable by spectroscopic, photochemical, biochemical, immunochemical, electrical, radiological, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish cites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence. Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

Amplification-Based Assays

In another embodiment, amplification-based assays can be used to measure copy number. In such amplification-based assays, the nucleic acid sequences act as a template in an amplification reaction (e.g. Polymerase Chain Reaction (PCR). In a quantitative amplification, the amount of amplification product will be proportional to the amount of template in the original sample. Comparison to appropriate (e.g. healthy tissue) controls provides' a measure of the copy number of the desired target nucleic acid sequence. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.).

Other suitable amplification methods include, but are not limited to ligase chain reaction (LCR) (see Wu and Wallace (1989) Genomics 4: 560, Landegren et al. (1988) Science 241: 1077, and Barringer et al. (1990) Gene 89: 117); transcription amplification (Kwoh et al. (1989) Proc. Natl. Acad. Sci. USA 86: 1173); and self-sustained sequence replication (Guatelli et al. (1990) Proc. Nat. Acad. Sci. USA 87: 1874).

Detection of Gene Expression

As indicated below, a number of cancer-associated nucleic acids are found in the regions of amplification disclosed here. Thus, cancer-associated genes can be detected by, for instance, measuring levels of the gene transcript (e.g. mRNA), or by measuring the quantity of translated protein.

Methods of detecting and/or quantifying gene transcripts using nucleic acid hybridization techniques are known to those of skill in the art (see Sambrook et al. supra). For example, a Northern transfer may be used for the detection of the desired mRNA directly. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target mRNA.

Alternatively, the gene transcript can be measured using amplification (e.g. PCR) based methods as described above for directly assessing copy number of the target sequences.

Detection of Expressed Protein

The “activity” of the cancer-associated nucleic acids can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

Therapeutic Methods

The invention provides a method for treating or alleviating a symptom of cancer in a subject by decreasing the expression or activity of an amplified cancer-associated nucleic acid or increasing expression or activity of a deleted cancer-associated gene. Therapeutic compounds are administered prophylactically or therapeutically to subject suffering from, or at risk of or susceptible to developing, cancer. Such subjects are identified using standard clinical methods.

The therapeutic method includes increasing the expression, or function, or both of one or more gene products of cancer-associated genes whose expression is decreased (“underexpressed”) in a subject or cell relative to a normal subject or cells of the same tissue type. In these methods, the subject is treated with an effective amount of a compound, which increases the amount of one of more of the underexpressed cancer-associated genes in the subject. Administration can be systemic or local. Therapeutic compounds include a polypeptide product of an underexpressed cancer-associated genes, or a biologically active fragment thereof, and a nucleic acid encoding an underexpressed cancer-associated gene and having expression control elements permitting expression in the subject. Administration of such compounds counters the effects of aberrantly-under expressed cancer-associated genes in the subject and improves the clinical condition of the subject

The method also includes decreasing the expression, or function, or both, of one or more gene products of cancer-associated genes whose expression is aberrantly increased (“overexpressed gene”) in cancer cells. Expression is inhibited in any of several ways known in the art. For example, expression is inhibited by administering to the subject a nucleic acid that inhibits, or antagonizes, the expression of the overexpressed cancer-associated gene or genes, e.g., an antisense oligonucleotide or siRNA which disrupts expression of the cancer-associated gene or genes.

Alternatively, function of one or more gene products of the overexpressed cancer-associated genes is inhibited by administering a compound that binds to or otherwise inhibits the function of the cancer-associated gene products. For example, the compound is an antibody which binds to the overexpressed gene product or gene products.

These modulatory methods are performed ax vivo or In vitro (e.g., by culturing the cell with the agent) or, alternatively, in vivo (e.g., by administering the agent to a subject). The method involves administering a protein or combination of proteins, a nucleic acid molecule or combination of nucleic acid molecules, or a combination of one or more nucleic acids and one or more proteins, as therapy to counteract aberrant expression or activity of the differentially expressed genes.

Diseases and disorders that are characterized by increased (relative to a subject riot suffering from the disease or disorder) levels or biological activity of the genes may be treated with therapeutics that antagonize (i.e., reduce or inhibit) activity of the overexpressed gene or genes. Therapeutics that antagonize activity are administered therapeutically or prophylactically.

Therapeutics that may be utilized include, e.g., (i) a polypeptide, or analogs, derivatives, fragments or homologs thereof, of the overexpressed or underexpressed sequence or sequences; (ii) antibodies to the overexpressed or underexpressed sequence or sequences; (iii) nucleic acids encoding the over or underexpressed sequence or sequences; (iv) antisense nucleic acids or nucleic acids that are “dysfunctional” (i.e., due to a heterologous insertion within the coding sequences of coding sequences of one or more overexpressed or underexpressed sequences); or (v) modulators (i.e., inhibitors, agonists and antagonists that alter the interaction between an over/underexpressed polypeptide and its binding partner. The dysfunctional antisense molecule is utilized to “knockout” endogenous function of a polypeptide by homologous recombination (see, e.g., Capecchi, Science 244: 1288-1292 1989). The siRNA is designed by methods known in the art to bind to gene transcripts and prevent translation into proteins.

Diseases and disorders that are characterized by decreased (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, a polypeptide (or analogs, derivatives, fragments or homologs thereof) or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods include contacting a cell with an agent that modulates one or more of the activities of the gene products of the under or over expressed genes. An agent that modulates protein activity includes a nucleic acid or a protein, a naturally-occurring cognate ligand of these proteins, a peptide, a peptidomimetic, or other small molecule. For example, the agent stimulates one or more protein activities of one or more of an under-expressed gene.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, nucleic acids for detecting the target sequences and other hybridization probes and/or primers. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### Pharmaceutical Preparations

The phrases “pharmaceutical” and “pharmacologically acceptable” refer to molecular entities and compositions that do not produce an adverse, allergic or other untoward reaction when administered to an animal, such as, for example, a human, as appropriate. The preparation of a pharmaceutical composition that contains at least one composition or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required within the industry.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, e.g., Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 μg/kg/body weight, about 5 μg/kg/body weight, about 10 μg/kg/body weight, about 50 μg/kg/body weight, about 100 μg/kg/body weight, about 200 μg/kg/body weight, about 350 μg/kg/body weight, about 500 μg/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 350 mg/kg/body weight, about 500 mg/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 mg/kg/body weight, etc., can be administered, based on the numbers described above.

The invention further provides a method of diagnosing a neoplasm, e.g., a solid tumor such as a breast, lung, colon, prostate or stomach tumor in a subject. A neoplasm is diagnosed by examining the copy number of gene loci from a test population of cells that contain a suspected tumor. The population of cells may contain the primary tumor, e.g., lung cancer, or may alternatively contain cells into which a primary tumor has disseminated, e.g., blood or lymphatic fluid. Preferably, the test cell population contains mostly cancer cells.

By “efficacious” is meant that the treatment leads to a decrease in size or metastatic potential of a neoplasm in a subject, or a shift in tumor stage to a less advanced stage. When treatment is applied prophylactically, “efficacious” means that the treatment retards or prevents a neoplasm from forming. Efficaciousness can be determined in association with any known method for treating a neoplasm. In some embodiments, the treatment is with an anti-tyrosine kinase agent, preferably imatinib, trastuzumab, erlotinib, or gefitinib.

Differences in the genetic makeup of individuals can result in differences in their relative abilities to metabolize various drugs. An agent that is metabolized in a subject to act as an anti-neoplastic agent can manifest itself by inducing a change in gene expression pattern in the subject's cells from that characteristic of a neoplastic state to a gene expression pattern characteristic of a non-neoplastic state. Accordingly, the differentially expressed tumor associated loci disclosed herein allow for a putative therapeutic or prophylactic anti-neoplastic agent to be tested in a test cell population from a selected subject in order to determine if the agent is a suitable anti-neoplastic agent in the subject.

To identify an anti-neoplastic agent that is appropriate for a specific subject, a test cell population from the subject is exposed to a therapeutic agent, and the expression of one or more of tumor associated sequences is measured.

The invention will be further described in the following examples, which do not limit the scope of the invention described in the claims. The following examples illustrate the identification and characterization of genes with a modified copy number, with or without associated changes in regulatory and/or coding sequences, in cancerous cells.

### EXAMPLES

Quantitative analysis of SNP array data from the cancer cell line samples revealed a variety of candidate copy number alterations, including both low-level and high-level amplifications, as well as hemizygous and homozygous deletions. Copy number analyses were similar regardless of whether the reference sample was paired normal DNA or pooled normal DNA.

### Example 1: General Methods

Primary Tumor and Cell Line Specimens

The following genomic DNA was obtained: lung adenocarcinoma (HOP-62, NCIH23), large cell lung carcinoma (LC) (HOP-92), and squamous cell lung carcinoma (NCI-H266) from the National Cancer Institute. Genomic DNA was prepared from the following cell lines: adenocarcinoma (H1437, H1819, H1993, H2009, H2087, H12122, H2347, HCC193, HCC461, HCC515, HCC78, HCC827), adenosquamous lung carcinoma (HCC366), LC (H2126, HCC1359, HCC1171), unspecified NSCLC (H2882, H2887), squamous cell lung carcinoma (H157, HCC15, HCC95), bronchioloalveolar carcinoma (BAC) (H358) and SCLC (H524, H526, H1184, H1607, H1963). The primary tumors were from anonymous patients and were surgically dissected and frozen at −80° C. until use. All primary tumor specimens were examined histologically to ensure at least 70% neoplastic tissue, except SCLC samples that were considered to have high tumor contents. These tumors consisted of 19 SCLC and 51 NSCLC. These include SCLC (S0168T, S0169T, S0170T, S0171T, S0172T, S0173T, S0177T, S0185T, S0187T, S0188T, S0189T, S0190T, S0191T, S0192T, S0193T, S0194T, S0196T, S0198T, S0199T), lung adenocarcinoma (MGH1622T, MGH7T, MGH1028T, S0356T, S0372T, S0377T, S0380T, S0392T, S0395T, S0397T, S0405T, S0412T, S0464T, S0471T, S0479T, S0482T, S0488T, S0498T, S0500T, S0502T, S0514T, S0522T, S0524T, S0534T, S0535T, S0539T, AD157T, AD163T, AD309T, AD311T, AD327T, AD330T, AD334T, AD335T, AD336T, AD337T, AD347T), squamous cell lung carcinoma (S0446T, S0449T, S0458T, S0465T, S0480T, S0485T, S0496T, S0508T, S0515T, S0536T), adenocarcinoma/BAC (S0376T), and BAC (S0509T, AD338T, AD362T).

SNP Array

For each sample, SNPs were genotyped with two different arrays, CentXba and CentHind, in parallel (Affymetrix, Inc., Santa Clara, Calif.). Array experiments were performed according to the manufacturer's recommendations. In brief, two aliquots of DNA (250 ng each) were first digested with XbaI or HindIII restriction enzyme (New England Biolabs, Boston, Mass.), respectively. The digested DNA was ligated to an adaptor before subsequent PCR amplification using AmpliTaq Gold (Applied Biosystems, Foster City, Calif.). Four 100 μl PCR reactions were then set up for each XbaI or HindIII adaptor-ligated DNA sample. The PCR products from four reactions were pooled, concentrated and fragmented with DNase I to a size range of 250 to 2000 bp. Fragmented PCR products were then labeled, denatured and hybridized to the array. After hybridization, the arrays were washed on the Affymetrix fluidics stations, stained and scanned using the Gene Chip Scanner 3000 and the genotyping software, Affymetrix Genotyping Tools Version 2.0.

Data Analysis

Data were normalized to a baseline array with median signal intensity at the probe intensity level with the invariant set normalization method described by Li et al. (30). After normalization, the signal values for each SNP in each array were obtained with a model-based (PM/MM) method (31). Signal intensities at each probe locus were compared with a set of normal reference samples, representing 12 individuals. From raw signal data, the inferred copy number at each SNP locus was estimated by applying the hidden Markovmodel (HMM) (27). The HMM model based on the assumption of diploidy or triploidy was applied; thus, possible normalized copy numbers are (0, 1, 2, 3, 4, . . . ; diploid) or (0, 0.67, 1.33, 2, 2.67, 3.33, 4, . . . ; triploid), leading to the possible copy number set (0, 0.67, 1, 1.33, 2, 2.67, 3, 3.33, 4, . . . ). The analysis methods described above are implemented in the dChip software Version 1.3, which is freely available to academic users (http://www.dchip.org). Mapping information of SNP locations and cytogenetic band is based on curation of Affymetrix Inc. (Santa Clara, Calif.) and University of California Santa Cruz hg 16 (http://genome.ucsc.edu).

The circular binary segmentation algorithm (32) was applied to the raw log 2 ratio data. This algorithm recursively splits chromosomes into subsegments based on a maximum t-statistic. The reference distribution for this statistic, estimated by permutation, is used to decide whether or not to split at each stage (see (32) for details).

The mean (rounded) raw estimated copy for each segment was compared to the HMM results.

Quantitative Real-Time PCR

Relative gene copy numbers and gene expression were determined by quantitative real-time PCR using a PRISM 7500 sequence Detection System (Applied Biosystem, Foster City, Calif.) and a QuantiTect SYBR Green PCR kit and a QuantiTect SYBR Green RT-PCR kit (Qiagen, Inc., Valencia, Calif.). The standard curve method was used to calculate target gene copy number in the tumor DNA sample normalized to a repetitive element Line-1 and normal reference DNA. The comparative threshold cycle method was used to calculate gene expression normalized to β-actin as a gene reference and normal human lung RNA as an RNA reference. Primers were designed using Primer 3 (http://frodo.wi.mit.edu/cgi-bin/primer3/primer3_www.cgi) and synthesized by Invitrogen (Carlsbad, Calif.). The primer sequences are shown below in Table B.

TABLE B

| | | | | | | Binery | | Representative | | | |
|---|---|---|---|---|---|---|---|---|---|---|---|
| | | | | | Mean dCHIP | segmentation | Real-time | gene | | | |
| | Start | Stop | Size | | copy | copy | PCR copy | within | Total # of | | |
| Cytoband ^{c} | (Mb) ^{c} | (Mb) ^{c} | (Mb) | Sample | number | number | number | interval | genes | Forward Seq | Reverse Seq |
| 1p342 | 39.51 | 40.55 | 1.04 | H1963 | 10.6 | 11 | 147.8 | MYCL.1 | 23 | TGCATGAAGCATTT | TGCAGCATCTCTCTCC |
| | | | | | | | | | | CCACAT (SEQ ID | AGAA(SEQ ID |
| | | | | | | | | | | NO: 1) | NO: 2) |
| | 39.55 | 40.91 | 1.36 | S0173T | 10.7 | 8 | 20.7 | | 24 | TGCATGAAGCATTT | TGCAGCATCTCTCTCC |
| | | | | | | | | | | CCACAT (SEQ ID | AGAA(SEQ ID |
| | | | | | | | | | | NO: 3) | NO: 4) |
| 2p24.3- | 14.2 | 16.38 | 2.18 | S0172T | 14.2 | 12 | 56.2 | MYCN | 9 | GTTCCTCCTCCAAC | GGTGCATCCTCACTCT |
| p24.2 | | | | | | | | | | ACCAAG (SEQ ID | CCAC (SEQ ID |
| | | | | | | | | | | NO: 5) | NO: 6) |
| | 15.25 | 17.06 | 1.81 | H526 | 7 | 5 | 42.3 | | 9 | GTTCCTCCTCCAAC | GGTGCATCCTCACTCT |
| | | | | | | | | | | ACCAAG | CCAC (SEQ ID |
| | | | | | | | | | | (SEQ ID NO: 7) | NO: 8) |
| 2q22.1 | 141.71 | 142.45 | 0.73 | H2122 | 0 | 0 | 0.01 | LRP1B | 1 | CCAGATCAATCAGG | TGACTGGCTCACTCGA |
| | | | | | | | | | | GTGACA (SEQ ID | AATCT (SEQ ID |
| | | | | | | | | | | NO: 9) | NO: 10) |
| | 141.79 | 142.78 | 0.99 | HCC95 | 0 | 1 | 0 | | 2 | GCAAGAAACACAGG | CTGGCTCCAAACAGCA |
| | | | | | | | | | | CAACAA (SEQ ID | ATTC (SEQ ID |
| | | | | | | | | | | NO: 11) | NO: 12) |
| | 141.94 | 142.2 | 0.26 | H2126 | 0 | 0 | 0 | | 1 | CCAGATCAATCAGG | TGACTGGCTCACTCGA |
| | | | | | | | | | | GTGACA (SEQ ID | AATCT (SEQ ID |
| | | | | | | | | | | NO: 13) | NO: 14) |
| | 142 | 142.2 | 0.2 | H157 | 0 | 0 | 0.06 | | 1 | CAGCTGCACTGGCA | TCTGCCTTGAATGACA |
| | | | | | | | | | | CTAGAC (SEQ ID | GTGTG (SEQ ID |
| | | | | | | | | | | NO: 15) | NO: 16) |
| 3p14.2 | 60.29 | 60.78 | 0.49 | HCC95 | 0 | 0 | 0 | FHIT | 4 | ATGCAAGCTATGCT | GGACCCACTGTGTTCT |
| | | | | | | | | | | CGGACT (SEQ ID | GTGA (SEQ ID |
| | | | | | | | | | | NO: 17) | NO: 18) |
| | 60.32 | 60.4 | 0.08 | H2887 | 0 | 0 | 0 | | 1 | ATGCAAGCTATGCT | GGACCCACTGTGTTCT |
| | | | | | | | | | | CGGACT (SEQ ID | GTGA (SEQ ID |
| | | | | | | | | | | NO: 19) | NO:20) |
| 3q25.1 | 152.82 | 152.95 | 0.12 | H2882 | 0 | 0 | 0.00* | AADAC, | 2 | TGGTTGGATTCATG | ACTTGCCCAGGAGAGA |
| | | | | | | | | SUCNR1 | | TCAGGAT (SEQ | ATCA (SEQ ID |
| | | | | | | | | | | ID NO: 21) | NO: 22) |
| | 152.82 | 152.95 | 0.12 | S0177T | 0 | 0 | 0.02* | | 2 | TGGTTGGATTCATG | ACTTGCCCAGGAGAGA |
| | | | | | | | | | | TCAGGAT (SEQ | ATCA (SEQ ID |
| | | | | | | | | | | ID NO: 23) | NO: 24) |
| 3q26.31- | 174.86 | 184.52 | 9.66 | S0465T | 7.8 | 5 | 10.29 | PIK3CA | 41 | TATTCGACAGCATG | ACTCCAAAGCCTCTTG |
| q27.1 | | | | | | | | | | CCAATC (SEQ ID | CTCA (SEQ ID |
| | | | | | | | | | | NO: 25) | NO: 26) |
| | 182.5 | 184.47 | 1.98 | S0515T | 13.2 | 11 | 309 | | 12 | TATTCGACAGCATG | ACTCCAAAGCCTCTTG |
| | | | | | | | | | | CCAATC (SEQ ID | CTCA (SEQ ID |
| | | | | | | | | | | NO: 27) | NO: 28) |
| 7p12.1- | 53.16 | 61.49 | 11.34 | HCC827 | 11.3 | 9 | 41.66 | EGFR | 50 | CCACCAAATTAGCC | CGCGACCCTTAGGTAT |
| q11.22 | | | | | | | | | | TGGACA (SEQ ID | TCTG (SEQ ID |
| | | | | | | | | | | NO: 29) | NO: 30) |
| | 54.24 | 69.62 | 9.73 | AD347T | 908 | 9 | 18.3 | | 123 | CCACCAAATTAGCC | CGCGACCCTTAGGTAT |
| | | | | | | | | | | TGGACA (SEQ ID | TCTG (SEQ ID |
| | | | | | | | | | | NO: 31) | NO: 32) |
| | 54.37 | 55.63 | 13.65 | S0480T | 13.7 | 10 | 47.84 | | 12 | CCACCAAATTAGCC | CGCGACCCTTAGGTAT |
| | | | | | | | | | | TGGACA (SEQ ID | TCTG (SEQ ID |
| | | | | | | | | | | NO: 33) | NO: 34) |
| 8p12- | 38.05 | 39.97 | 1.93 | MGH1622T | 10.4 | 11 | 14.92 | FGFR1 | 22 | CCAGGGCTGGAATA | CTTGGAGGCCAGATAC |
| p11.22 | | | | | | | | | | CTGCTA SEQ ID | TCCA (SEQ ID |
| | | | | | | | | | | NO: 35) | NO: 36) |
| | 38.73 | 39.84 | 1.11 | S0449T | 6.4 | 5 | 6.07 | | 11 | CCAGGGCTGGAATA | CTTGGAGGCCAGATAC |
| | | | | | | | | | | CTGCTA (SEQ ID | TCCA (SEQ ID |
| | | | | | | | | | | NO: 37) | NO: 38) |
| 8q24.13- | 126.6 | 128.89 | 2.3 | H524 | 6.6 | 5 | 174.51 | MYC | 6 | CCAGAGGAGGAACG | TTGGACGGACAGGATG |
| q24.21 | | | | | | | | | | AGCTAA (SEQ ID | TATG (SEQ ID |
| | | | | | | | | | | NO: 39) | NO: 40) |
| | 127.46 | 128.89 | 1.43 | HCC827 | 6.9 | 6 | 8.63 | | 6 | CCAGAGGAGGAACG | TTGGACGGACAGGATG |
| | | | | | | | | | | AGCTAA (SEQ ID | TATG (SEQ ID |
| | | | | | | | | | | NO: 41) | NO: 42) |
| | 127.59 | 130.83 | 3.24 | NCI-H23 | 8 | 7 | 11.11 | | 11 | CCAGAGGAGGAACG | TTGGACGGACAGGATG |
| | | | | | | | | | | AGCTAA (SEQ ID | TATG (SEQ ID |
| | | | | | | | | | | NO: 43) | NO: 44) |
| | 127.9 | 129.62 | 1.72 | H2122 | 3.6 | 5 | 14.49 | | 6 | CCAGAGGAGGAACG | TTGGACGGACAGGATG |
| | | | | | | | | | | AGCTAA (SEQ ID | TATG (SEQ ID |
| | | | | | | | | | | NO: 45) | NO: 46) |
| | 128.44 | 129.6 | 1.16 | H2087 | 7.9 | 8 | 15.99 | | 4 | CCAGAGGAGGAACG | TTGGACGGACAGGATG |
| | | | | | | | | | | AGCTAA (SEQ ID | TATG (SEQ ID |
| | | | | | | | | | | NO: 47) | NO: 48) |
| 9p23 | 8.61 | 9.12 | 0.51 | S077T | 0 | 0 | 0.01 | PTPRD | 2 | GTGCGGAGGAAGAA | GAGGCAGTGATTCCAA |
| | | | | | | | | | | AGTGAG (SEQ ID | GCTG (SEQ ID |
| | | | | | | | | | | NO: 49) | NO: 50) |
| | 8.79 | 9.55 | 0.77 | H358 | 0 | 0 | 0.00* | | 2 | TCTAGCTGCTGGCA | GCTGAAGGAAGCCTGT |
| | | | | | | | | | | CTGGTA (SEQ ID | GTTC (SEQ ID |
| | | | | | | | | | | NO: 51) | NO: 52) |
| | 9.41 | 9.61 | 0.2 | HCC1171 | 0 | 0 | 0.08* | | 1 | ACGAGGTGTGTGGT | AGTTGATGCCAGCTCC |
| | | | | | | | | | | GTTCAA (SEQ ID | ATGT (SEQ ID |
| | | | | | | | | | | NO: 53) | NO: 54) |
| | 9.5 | 9.75 | 0.25 | H2347 | 0 | 0 | 0.00* | | 1 | ACGAGGTGTGTGGT | AGTTGATGCCAGCTCC |
| | | | | | | | | | | GTTCAA (SEQ ID | ATGT (SEQ ID |
| | | | | | | | | | | NO: 55) | NO: 56) |
| 9p21.3 | 20.9 | 22.94 | 2.03 | H2126 | 0 | 0 | 0 | CDKN2A | 35 | GCGCTACCTGATTC | CAACGCACCGAATAGT |
| | | | | | | | | | | CAATTC (SEQ ID | TACG (SEQ ID |
| | | | | | | | | | | NO: 578) | NO:58) |
| | 21.2 | 22.19 | 0.98 | HCC1359 | 0 | 0 | 0.01 | | 21 | GCGCTACCTGATTC | CAACGCACCGAATAGT |
| | | | | | | | | | | CAATTC (SEQ ID | TACG (SEQ ID |
| | | | | | | | | | | NO: 59) | NO: 60) |
| | 21.58 | 25.1 | 3.52 | HCC1171 | 0 | 0 | 0 | | 11 | GCGCTACCTGATTC | CAACGCACCGAATAGT |
| | | | | | | | | | | CAATTC (SEQ ID | TACG (SEQ ID |
| | | | | | | | | | | NO: 61) | NO: 62) |
| | 21.7 | 23.39 | 1.69 | H2882 | 0 | 1 | 0 | | 6 | GCGCTACCTGATTC | CAACGCACCGAATAGT |
| | | | | | | | | | | CAATTC (SEQ ID | TACCG (SEQ ID |
| | | | | | | | | | | NO: 63) | NO: 64) |
| | 21.84 | 26.83 | 4.99 | HCC95 | 0 | 0 | 0 | | 11 | GCGCTACCTGATTC | CAACGCACCGAATAGT |
| | | | | | | | | | | CAATTC (SEQ ID | TACG (SEQ ID |
| | | | | | | | | | | NO: 65) | NO: 66) |
| | 21.95 | 22.09 | 0.14 | H2122 | 0 | 0 | 0.01 | | 3 | GCGCTACCTGATTC | CAACGCACCGAATAGT |
| | | | | | | | | | | CAATTC (SEQ ID | TACG (SEQ ID |
| | | | | | | | | | | NO: 67) | NO: 68) |
| | 24.34 | 24.7 | 0.36 | H157 | 0 | 0 | 0.03* | | 1 | GGCAGAGCTGAAGT | AGGTATCAGCAAGCAA |
| | | | | | | | | | | GGAAAC (SEQ ID | TTGGA (SEQ ID |
| | | | | | | | | | | NO: 69) | NO: 70) |
| 10q23.31 | 89.03 | 89.4 | 0.37 | H1607 | 0 | 0 | 0 | PTEN | 4 | ATGTGGCGGGACTC | AGCGGCTCAACTCTCA |
| | | | | | | | | | | TTTATG (SEQ ID | AACT (SEQ ID |
| | | | | | | | | | | NO: 71) | NO: 72) |
| | 89.18 | 89.88 | 0.69 | S0187T | 0 | 0 | 0.09 | | 4 | ATGTGGCGGGACTC | AGCGGCTCAACTCTCA |
| | | | | | | | | | | TTTATG (SEQ ID | AACT (SEQ ID |
| | | | | | | | | | | NO: 73) | NO: 74) |
| | 89.35 | 91.16 | 1.8 | S0189T | 0 | 0 | 0.12* | | 22 | TGGGCAGAGTGAAA | TAGCATGTGTTCGCCC |
| | | | | | | | | | | TCATCA (SEQ ID | ATAA (SEQ ID |
| | | | | | | | | | | NO: 75) | NO: 76) |
| 12p11.21 | 32.17 | 33.02 | 0.85 | S0515T | 8.8 | 7 | 10.75 | PKP2 | 6 | TCCTGACACACAGT | GTTTAGAAGGTCGCGT |
| | | | | | | | | | | CCCGAGTA (SEQ | GCAT (SEQ ID |
| | | | | | | | | | | ID NO: 77) | NO:78) |
| | 32.69 | 36.59 | 3.9 | H2087 | 7.8 | 8 | 11.43 | | 12 | TCCTGACACACAGT | GTTTAGAAGGTCGCGT |
| | | | | | | | | | | CCCGAGTA (SEQ | GCAT (SEQ ID |
| | | | | | | | | | | ID NO: 79) | NO: 80) |
| 12q13.3- | 56.26 | 57.37 | 1.1 | H2087 | 8.8 | 9 | 23.4 | CDK4 | 20 | TCTGATGCGCCAGT | TTCCACCACTTGTCAC |
| q14.1 | | | | | | | | | | TTCTAA (SEQ ID | CAGA (SEQ ID |
| | | | | | | | | | | NO: 81) | NO: 82) |
| | 55.82 | 56.67 | 0.85 | HCC827g | 13 | 13 | 30.34 | | 34 | TCTGATGCGCCAGT | TTCCACCACTTGTCAC |
| | | | | | | | | | | TTCTAA (SEQ ID | CAGA (SEQ ID |
| | | | | | | | | | | NO: 83) | NO: 84) |
| 19q12 | 34.02 | 35.55 | 1.53 | S0524T | 6.7 | 7 | 6.77 | CCNE1 | 12 | AAGTGGATGGTTCC | CAAATCCAAGCTGTCT |
| | | | | | | | | | | ATTTGC (SEQ ID | CTGTG (SEQ ID |
| | | | | | | | | | | NO: 85) | NO: 86) |
| | 34.79 | 37.09 | 2.3 | S0188T | 7.9 | 7 | 10.93 | | 12 | AAGTGGATGGTTCC | CAAATCCAAGCTGTCT |
| | | | | | | | | | | ATTTGC (SEQ ID | CTGTG (SEQ ID |
| | | | | | | | | | | NO: 87) | NO: 88) |
| 22q11.21- | 16.99 | 20.31 | 3.32 | H1819 | 6.8 | 7 | 12.57 | CRKL | 92 | CGGAAAGCATGGAA | AACCGGAAACTGCAGG |
| q11.22 | | | | | | | | | | ATAGGA (SEQ ID | TAGA (SEQ ID |
| | | | | | | | | | | NO: 89) | NO: 90) |
| | 17.51 | 21.44 | 3.93 | HCC515 | 7.4 | 7 | 14.01 | | 169 | CGGAAAGCATGGAA | AACCGGAAACTGCAGG |
| | | | | | | | | | | ATAGGA (SEQ ID | TAGA (SEQ ID |
| | | | | | | | | | | NO: 91) | NO: 92) |
| | 18.47 | 20.61 | 2.14 | S0380T | 6.4 | 5 | 8.44 | | 68 | CGGAAAGCATGGAA | AACCGGAAACTGCAGG |
| | | | | | | | | | | ATAGGA (SEQ ID | TAGA (SEQ ID |
| | | | | | | | | | | NO: 93) | NO: 94) |
| | 19.45 | 20.75 | 1.29 | HCC1359 | 6.5 | 7 | 8.05 | | 48 | CGGAAAGCATGGAA | AACCGGAAACTGCAGG |
| | | | | | | | | | | ATAGGA (SEQ ID | TAGA (SEQ ID |
| | | | | | | | | | | NO: 95) | NO: 96) |

| | forward | reverse |
|---|---|---|
| LINE1 | AAAGCCGCTCAACTACATGG | TGCTTTGAATGCGTCCCAGAG |
| | (SEQ ID NO: 97) | (SEQ ID NO: 98) |
| ACTB | AAATCTGGCACCACACCTTC | CAGAGGCGTACAGGGATAGC |
| | (SEQ ID NO: 99) | (SEQ ID NO: 100) |
| ASPH | AAGGCTGCAAGATTCGATGT | TATCAGCCGGAAAGATGAGG |
| | (SEQ ID NO: 101) | (SEQ ID NO: 102) |
| MGC34646 | GCAATGCACGTGAAGCATAC | GAGTGGCTGGGTGTTCTCAT |
| | (SEQ ID NO: 103) | (SEQ ID NO: 104) |
| CRKL | CCTGTCTTTGCGAAAGCAAT | TGACTTTCACGATGTCACCAA |
| | SEQ ID NO: 105) | (SEQ ID NO: 106) |
| PIK4CA | AAGAAAGCACAGCTCGGAAA | TAGGCCACATCAGACAGCAG |
| | (SEQ ID NO: 107) | (SEQ ID NO: 108) |

Interphase FISH

FISH probes were made from BAC clones RP11-805B16 and RP11-153K21 (Children's Hospital, Oakland Research Institute, Oakland, Calif.), identified to overlap the ASPH locus. BAC DNA was purified and 100 ng of each clone was labeled with Digoxigene-dUTP using random primers. The DNA was then purified with a MicroSpin S-200 HR Column, ethanol precipitated and resuspended in 100 μl hybridization solution. The control FISH probe, CEP 8 SpectrumOrange (Vysis, Downers Grove, Ill.), detects the centromeric region of chromosome 8. H2171 cells were grown in culture using standard methods and harvested by centrifugation after 3 days of growth. Slides for FISH analysis were prepared according to the control probe manufacturer's directions (Vysis, Downers Grove, Ill.). Briefly, cell pellets were resuspended in a fixative, a 3:1 solution of methanol and acetic acid. The cell suspension was dropped onto slides dipped in fixative and dried for 10 minutes over a 67° C. water bath. The slides were pretreated with 2×SSC (salt sodium citrate) at 37° C. for 1 hour, then digested for 5 minutes with a 1:25 solution of All III and rinsed with PBS (phosphate buffered saline). They were then incubated for 1 min in 10% buffered formalin at room temperature, rinsed with PBS, dehydrated in an ethanol series (70, 85, 95 and 100%) and air dried. 10 μl of probe solution (6l hybridization buffer, 1 μl Cot-1 DNA, 1μl centromere control probe and 1μl each of RP11-805B16 and RP11-153K21 probes) was incubated on the slide under a scaled coverslip for 3.5 minutes at 85° C. and then placed in a humidified chamber overnight at 37° C. The slides were then washed in 0.5×SSC at 75° C. for 5 minutes. The ASPH probes were detected using a 1:500 dilution of FITC anti-Digoxigenin in 10% normal Goat Serum, with DAPI as a counterstain.

### Example 2: Genome-Wide Analysis of Lung Cancer

A total of 101 human lung carcinoma DNA samples, including 51 NSCLC primary tumor samples, 26 NSCLC cell line samples, 19 SCLC primary tumor samples, and 5 SCLC cell line samples, were hybridized to SNP arrays containing 115,593 mapped SNP loci. Two independent algorithms, the hidden Markov model (HMM) in dChipSNP (27) and binary segmentation analysis (32), were used to infer copy number and thereby to identify genomic amplifications and deletions.

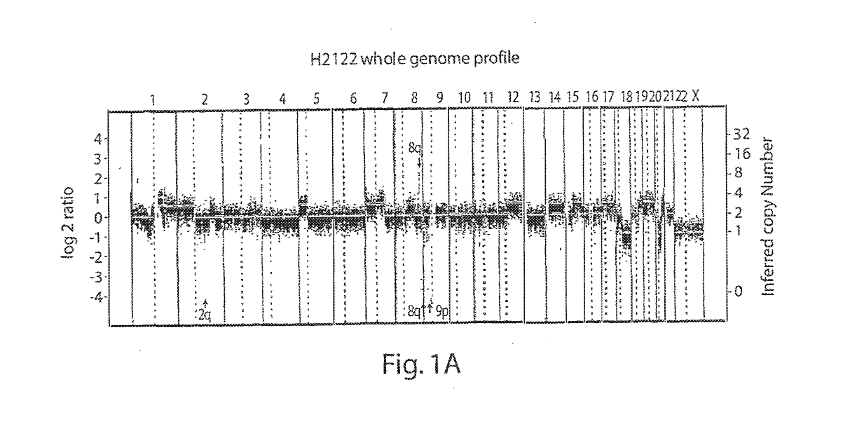

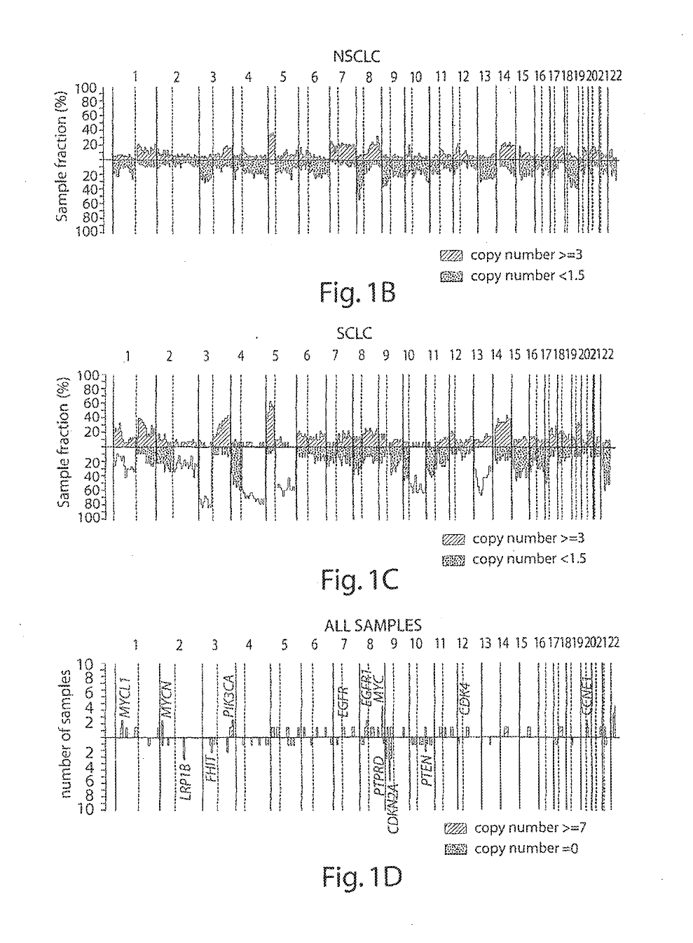

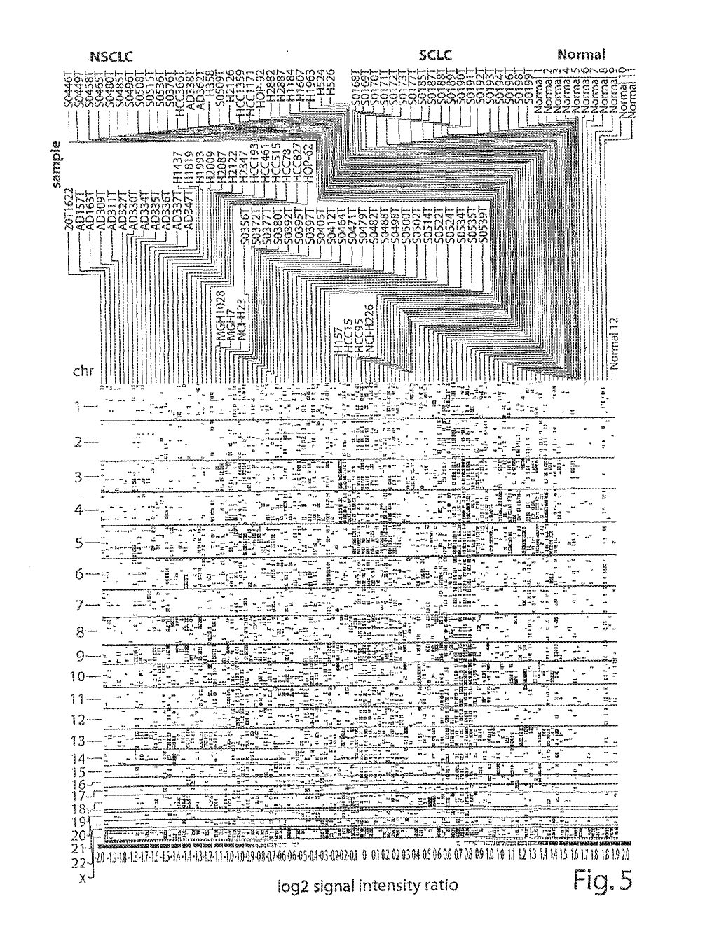

The genomes of lung carcinomas are often complex, and numerous chromosomal alterations can be seen in many samples. An example is shown in FIG. 1A; the lung adenocarcinoma cell line H2122 shows homozygous deletions on chromosome arms 2q, 8q, and 9p, as well as a significant amplification on chromosome arm 8q (FIG. 1A). The pattern of copy number gains and losses, across all samples and throughout the genome identifies recurrent regions of copy number alteration in lung carcinomas (FIG. 1B, C; FIG. 5 shows the pattern of copy number alterations for each sample across the entire genome). In both NSCLC and SCLC, the most frequent copy number gains were found in chromosome arm 5p, with >=3 copies found in 25% and 43% of samples, respectively (FIG. 1B,C). However, the region of 5p copy number gain is usually large, and no area of focal amplification could be identified from this data set.

Maximum degrees of copy number loss at a given locus, with an inferred copy number <1.5 based on HMM analysis were found most often in chromosome arms 8p (33%) and 9p (26%) in NSCLC (FIG. 1B), and in chromosome arms 3p (68%) and 4q (58%) in SCLC (FIG. 1C). These findings are broadly similar to results reported by CGH (33) but overall we are reporting a somewhat lower frequency of these chromosome alterations. One of the reasons may be the presence of stromal admixture in primary tumor samples, a second reason may be that copy number signal is attenuated somewhat in this analysis, whether in terms of primary hybridization intensity or the application of the HMM, and a third possible reason is that maximal regions of loss rather than tallying the presence of loss within a whole chromosome arm are analyzed. For example, the SCLC cell lines NCI-H524 and NCI-H526 only show partial loss of chromosome 3p in SNP array analysis (FIG. 5); similar results are seen for the same cell lines upon analysis with cDNA array CGH

The large regions of modest copy gain and loss shown in FIGS. 1B and 1C do not determine the presence or absence of alterations in specific genes within these regions.

Homozygous deletions and amplifications are of particular interest because they may indicate tumor suppressor gene and oncogene loci, respectively. Regions of homozygous deletion were defined as segments of at least 4 SNP loci covering >5 kb with an inferred copy number of 0 according to the hidden Markov model (HMM) described in the Materials and Methods. Similarly, regions of high-level amplification were defined as segments having at least 4 SNP loci covering >5 kb with an inferred copy number >=7.

The amplifications and homozygous deletions recurrent in multiple samples were verified by real-time PCR (Table 1). In general, copy number estimation was consistent between the HMM and binary segmentation approaches. On average, the number of annotated genes is greater for regions of recurrent, high level amplification (copy number >=7) than for recurrent homozygously deleted regions (14.6 vs. 7.7 genes/Mb, respectively). Given the parameters used in these experiments, the HMM algorithm was able to identify several amplified regions that were not found by binary segmentation but were verified by real-time PCR analysis. (All homozygous deletions and high-level amplifications identified in this study are shown in Tables 3 and 4, respectively.)

The frequencies of high-level amplification (copy number >=7) and homozygous deletion (copy number=0) across the genome in NSCLC and SCLC are displayed in FIG. 1D. A total of six distinct recurrent homozygous deletions were identified (Table 1 and FIG. 1D, blue bars below the line). The most common homozygous deletion (7 out of 26 NSCLC lines) is in the cyclin-dependent kinase inhibitor gone, CDKN2A, on chromosome 9p21, well-known to be deleted in non-small cell lung carcinoma. Other deletions comprise the phosphatase and tensin homolog PTEN tumor suppressor gene on chromosome 10q23, in 1 out of 5 SCLC lines and 2/19 primary tumors, and the FHIT gene on chromosome 3p, also primarily in SCLC; 3p allele loss is believed to be the most frequent and earliest genetic alteration in the multistage development and progression of lung cancer (34). A homozygous deletion of chromosome 2q22.1 was found in four out of 26 NSCLC cell lines (Table 1). Each of these four deletions fall within a single known gene, the low density lipoprotein-related protein 1B gene (LRP1B), but in no case is the entire LRP1B gene deleted; it is unknown whether LRP1B or some undescribed transcript is the target of these deletions. This gene has been previously identified as a candidate tumor suppressor gene, with ˜17% of NSCLC lines showing homozygous deletions (35). Interestingly, every cell line with interstitial LRP1B deletion also has undergone complete deletion of CDKN2A (Table 1). Finally, two recurrent homozygous deletions on chromosome 3q25 and 9p23 were identified (see below).

Genes that most frequently undergo copy number gain (copy number >=4) include the Myc family members MYC (4 out of 26 NSCLC cell lines, 2/37 adenocarcinoma tumors and 3/24 of SCLC lines and tumors), MYCL1 (5/24 SCLC lines and tumors and 1/10 squamous tumors), and MYCN (3/24 SCLC lines and tumors and 1/12 adenocarcinoma tumors) (36-38); regions encompassing the EGFR (5/49 adenocarcinoma cell lines and tumors and 1/10 squamous tumors) (39), FGFR1 (3/13 squamous cell lines and tumors and 3/64 non-squamous NSCLC lines and tumors), and CDK4 (6/49 adenocarcinoma cell lines and tumors) (40) kinase genes and the CCNE1 (1/19 SCLC tumors, 1/37 adenocarcinoma tumors and 1/26 NSCLC lines) cyclin gene; and the PIK3CA gene (4/13 squamous cell lines and tumors, 3/19 SCLC tumors and 3/64 non-squamous NSCLC lines and tumors (18) (Table 1 and FIG. 1D, red bars above the line). All of these amplifications have been previously reported in lung carcinoma except FGFR1 and CCNE1, which have been reported in other tumor types (41-45).

The 8q12-13 locus, is amplified in a second SCLC sample in this study. Two novel amplicons on chromosome 12p11 and 22q11 were also identified.

### Example 3: Homozygous Deletions within Chromosome Segments 3Q25 and 9P23

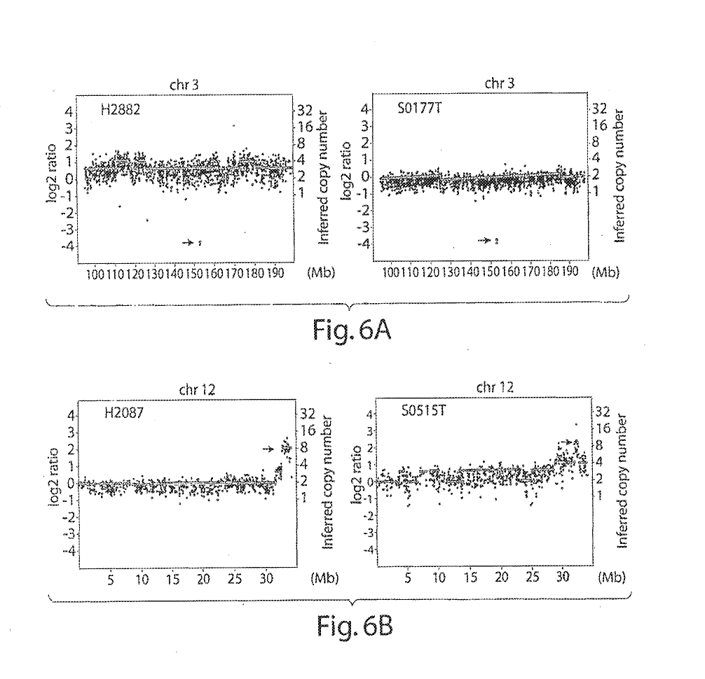

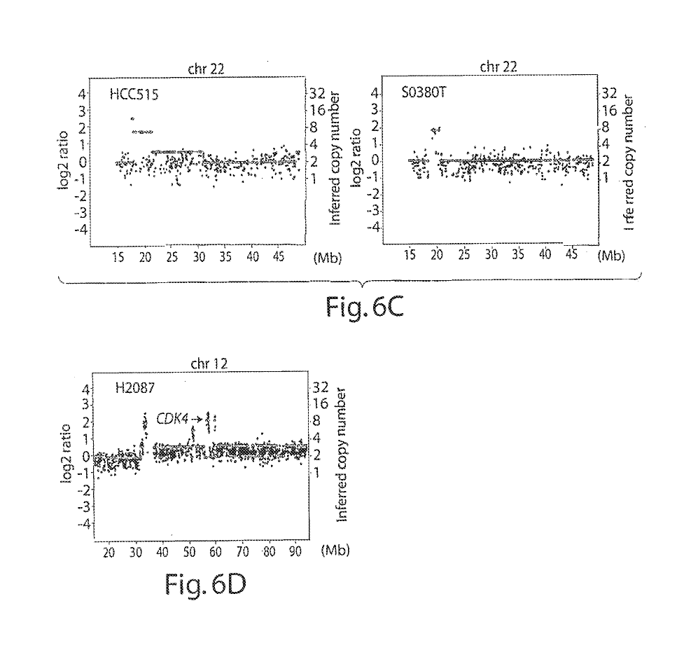

Two samples, the H2882 cell line (NSCLC) and the S0177T primary tumor (SCLC), showed homozygous deletions on 3q25 (Table 1, FIG. 6A). This 120 kb region included only two genes, AADAC and SUCNR1. AADAC encodes arylacetamide deacetylase, which is predicted to catalyze biotransformation pathways for arylamine and heterocyclic amine carcinogens. SUCNR1 is a G-protein coupled receptor for the citric acid cycle intermediate, succinate and may be involved in succinate-induced hypertension (46). Both deletions were confirmed by real time quantitative PCR analysis of genomic DNA (Table 1).

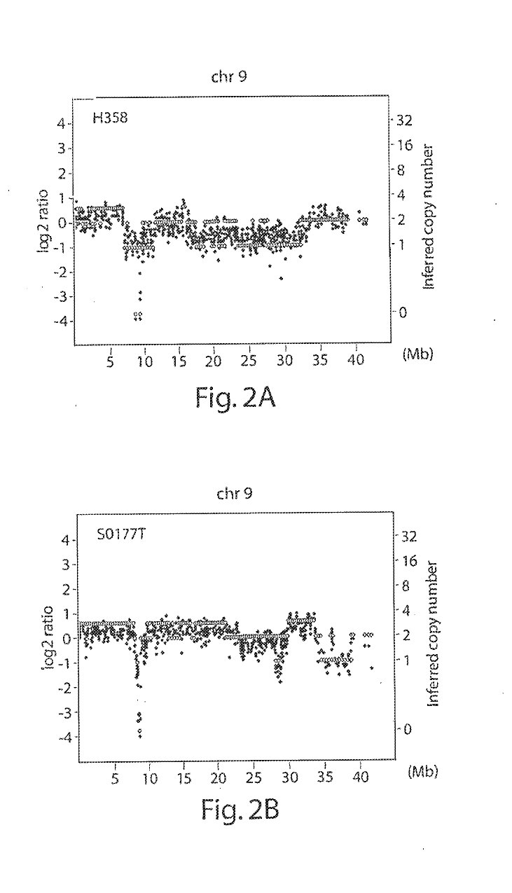

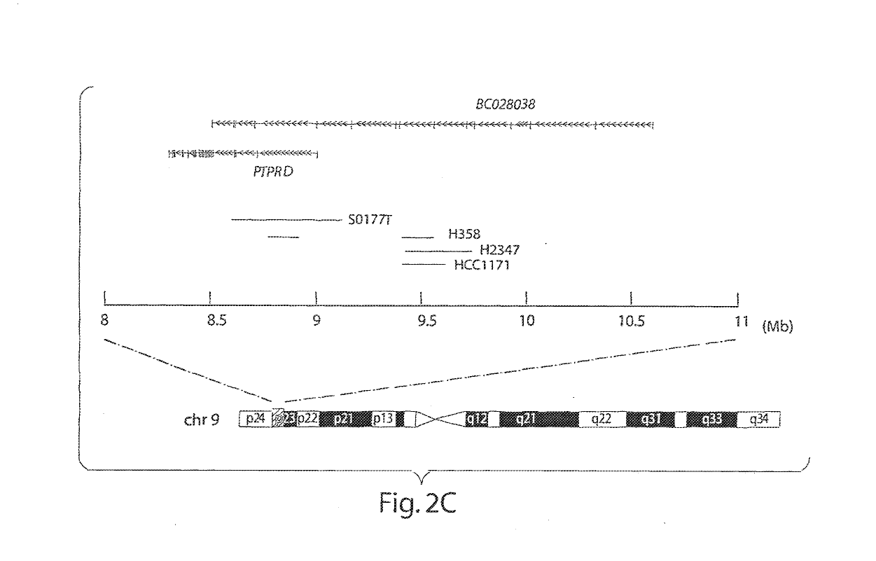

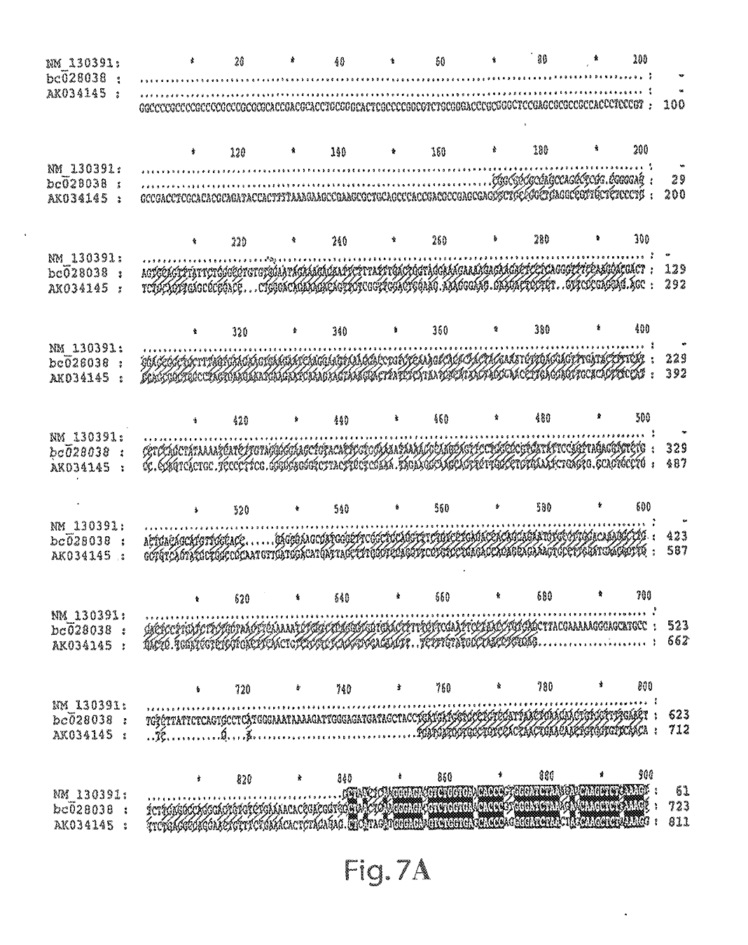

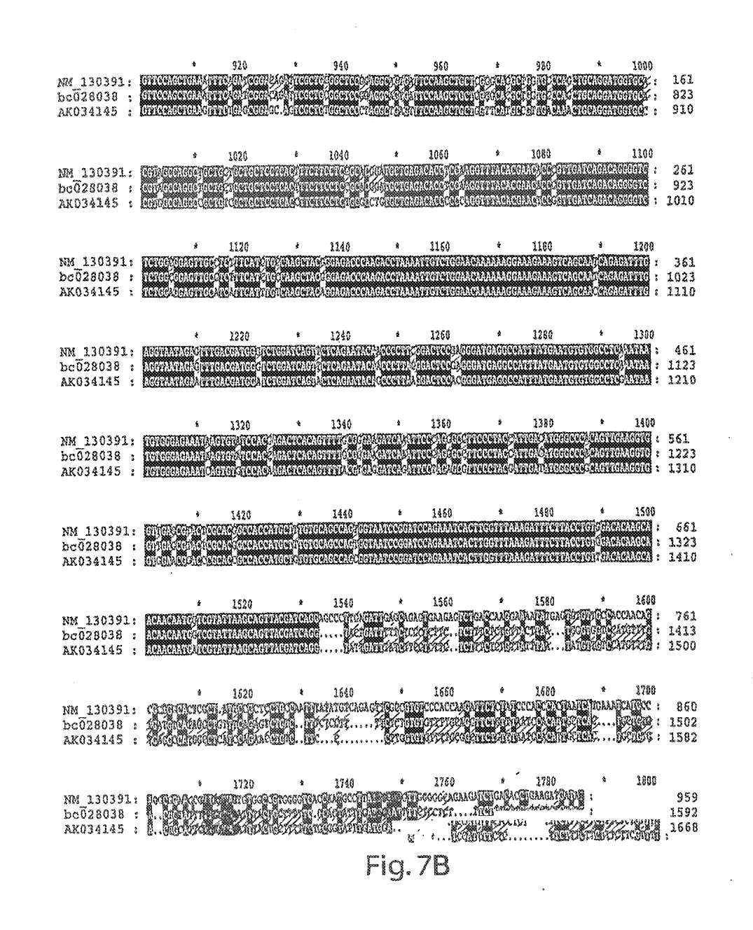

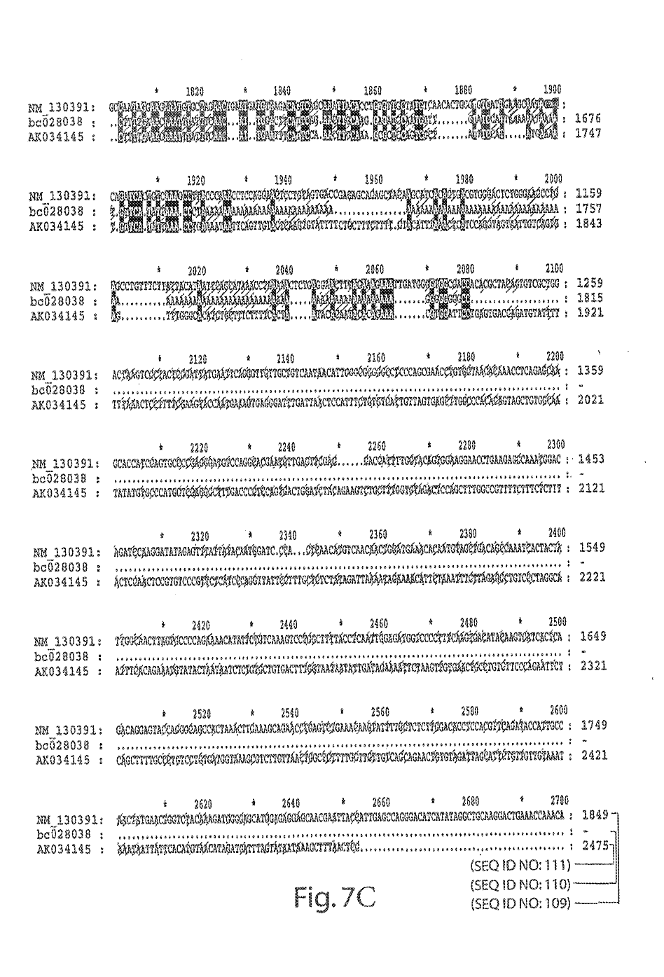

Chromosome 9p undergoes frequent LOH in lung and other cancers, typically associated with homozygous deletion or other inactivation of CDKN2A. This data has identified an additional region of homozygous deletion on chromosome 9p23-24.1, telomeric to CDKN2A, which includes sequence upstream of and in the 5′-most portion of the protein tyrosine phosphatase, receptor type, D (PTPRD) gene (FIG. 2A, B; Table 1). One SCLC primary tumor, S0177T, and one NSCLC cell line, H358, contained homozygous deletions confirmed by real-time PCR upstream of PTPRD and in the 5′ UTR, exon 1, and intron 1 of this gene (FIG. 2C). Two additional cell lines, H2347 and HCC1171, as well as H358, also contain homozygous deletions further upstream of the PTPRD coding region and more centromeric on chromosome 9 (FIG. 2C and Table 1). While not all of the deletions remove exons of PTPRD, all of the homozygous deletions eliminate exons from an uncharacterized spliced transcript whose sequence and exonic structure are conserved between human and mouse (BC028038; corresponds to the position ˜8.52-10.60 Mb on human chromosome 9). This transcript contains a unique 5′ end, several central exons shared with PTPRD, and a unique 3′ end. An alignment of PTPRD, BC028038 and the similar mouse transcript is shown in FIG. 7. The role for PTPRD or the BC028038 transcript in lung tumorigenesis has not been determined. The frequency of alterations in these genes in lung cancer, will be determined by further SNP array analysis, by quantitative PCR, and by sequencing.

### Example 4: 8Q Amplification in Small Cell Lung Carcinoma

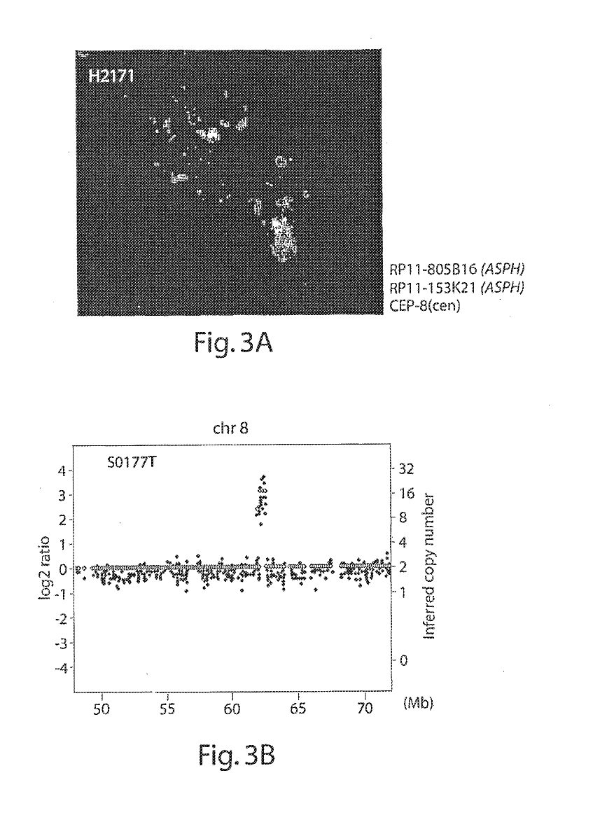

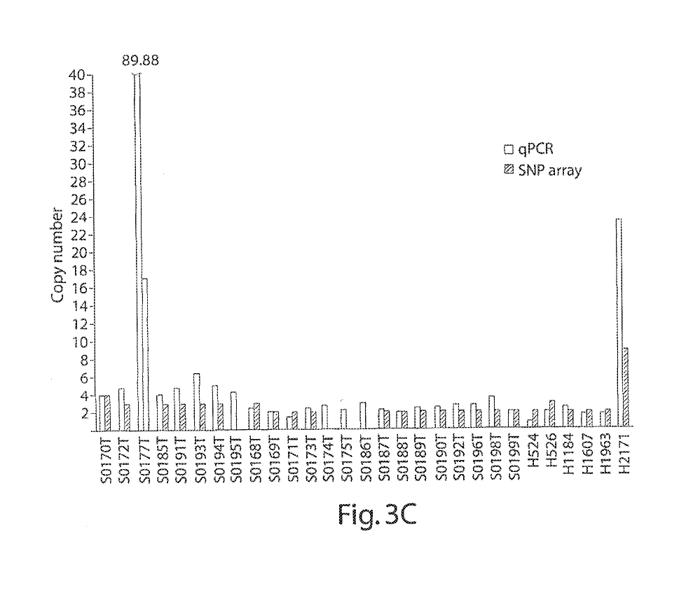

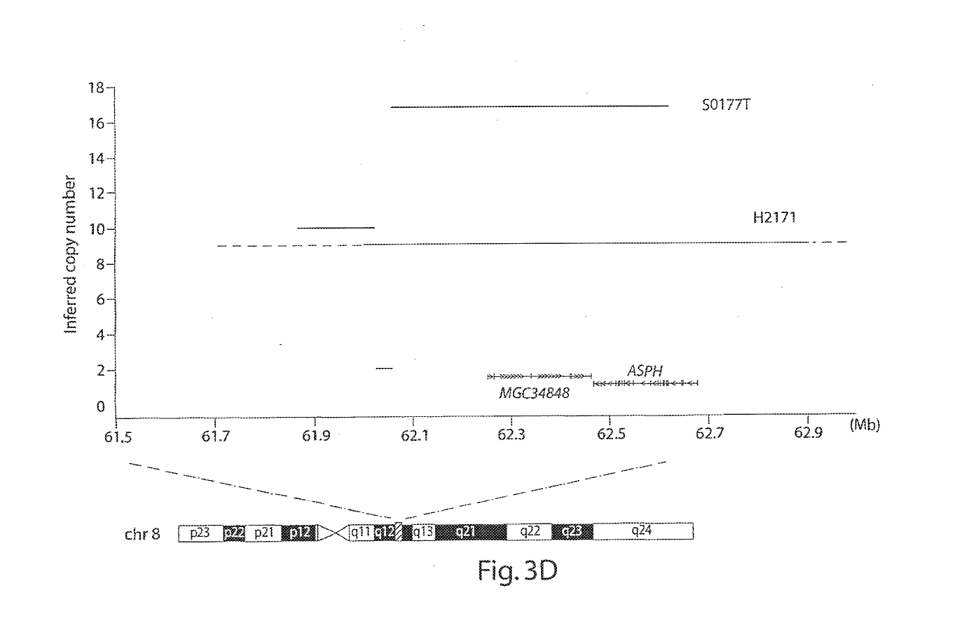

A high-level amplicon of chromosome 8q12.1-q13.11, 1.7-2.6 Mb in size in the SCLC cell line H2171, was identified in our previous study using SNP arrays representing ˜10,000 SNP loci (27). Interphase FISH analysis on the H2171 line confirmed the amplification of the 8q12-13 locus, with an estimated copy number of at least 12-20 (FIG. 3A). In this study, SNP array analysis of one small cell lung cancer primary tumor sample, S0177T, revealed high-level amplification of the 8q12-13 region near the ASPH locus (FIG. 3B). Quantitative real-time PCR revealed a copy number of 89.9, a 45-fold increase over normal genomic DNA. SNP array analysis revealed lower level copy number gains (>=4) of the 8q12-13 region in additional SCLC primary tumors, which were verified by quantitative real-time PCR (FIG. 3C) and in 3 out of 22 NSCLC cell lines.

The primary SCLC tumor sample, S0177T, represents both the smallest extent of the 8q12-13 amplicon, 670-750 kb in size, and the largest amplitude for copy number gain of all samples tested. Interestingly, this amplicon does not contain the entire ASPH gene, but does include its catalytic domain and one additional open reading frame, MGC34646, encoding a protein containing a Sec14p-like lipid-binding domain (FIG. 3D). Real time reverse transcriptase PCR analysis showed the relative expression of ASPH was 12 fold higher in H2171 cell line than in normal lung. Quantitative PCR analysis of MGC34646 expression revealed it to be greater than 100-fold higher in H2171 than in control SCLC cell lines that do not have 8q12-13 amplification (not shown); MGC34646 expression was not detectable from normal lung tissue. These data suggest that MGC34646 may be a target of chromosomal amplification resulting in significantly increased gene expression.

### Example 5: Recurrent Amplification on 12P11 and 22Q11 in Non-Small Cell Lung Carcinoma

Amplification of 12p11 was found in two NSCLC samples (Table 1, FIG. 6B), with an overlapping region from 32.7 to 33 Mb. The minimally amplified region contains 4 genes, LOC283343, a pseudogene similar to argininosuccinate synthetase, and CGI-04, a provisional protein coding gene, as well as DNM1L and PKP2. DNM1L encodes dynamin like protein 1, which is a member of the dynamin family of GTPases, involved in the fission of organelles such as mitochondria and peroxisomes (47, 48). PKP2 encodes plakaphilin 2, which may be involved in betacatenin signaling (49).

Two adenocarcinoma cell lines, HCC515 and H1819, one primary adenocarcinoma tumor, S0380T, and one large cell carcinoma cell line, HCC1359, showed high-level amplification of 22q11 (Table 1), with a minimal region of overlap from 19.45-20.31 Mb (Table 5). Examples from the HCC515 cell line and S0380T primary tumor are shown in FIG. 6C. High-level amplification on 22q11 has not been previously described in lung cancer. The target gene within the 22q11 amplicon has not yet been identified, but genes of interest that map to this region include CRKL and PIK4CA. CRKL is a member of the CRK adapter protein family, which includes homologs of the v-CRK oncogene (50). PIK4CA is the catalytic subunit of phosphatidylinositol 4-kinase alpha, which is responsible for the downstream production of certain cell signaling molecules. The high level amplification of the 22q11 region was confirmed by real time quantitative PCR analysis of genomic DNA (Table 1). Real time quantitative reverse transcriptase PCR analysis of cell line-derived mRNA showed the relative expression of CRKL in cells with 22q11 amplification to be higher (5.32 fold in HCC515 and 3.75 fold in HCC1359) than PIK4CA expression (2.61 fold in HCC515 and to be 0.4 fold higher in HCC1359), compared to both normal human lung and lung cancer lines without 22q11 amplification. However, a significant increase in CRKL protein expression was not found in cell lines containing the amplicon. Thus the target of the amplification remains unknown.

### Example 6: Copy Number Aberrations in the CDK4/CDK6 Pathway in NSCLC

The RB/p16/CDK4/CDK6 pathway is often disrupted in tumorigenesis. Copy number alterations of CCND1, CDK4, CDK6, p16, RB1 as well as CCNE1 were present in this data and were for the most part non-overlapping, as expected with genes in a pathway. However, clear target genes have yet to be identified, as these regions with copy number alterations contain several genes in addition to these candidate ones. The CDK2NA locus on chromosome 9p21 is frequently subject to homozygous deletion in NSCLC (51) as in many other cancers. Seven NSCLC cell lines in this study were found to undergo loss of both copies of the CDKN2A locus (Table 1), confirming frequent deletion of this region; it is suspected that there were also primary tumors with homozygous deletion but this finding can not be confirmed in the face of stromal admixture.

Cyclin D1 (CCND1) amplification and overexpression has been previously described in NSCLC (52). The region containing the cyclin D1 gene CCND1 was amplified in five NSCLC cell lines (Table 5). High-level amplification (3 to 4-fold) of the region surrounding the cyclin E (CCNE1) gene (19q12) also was present in two primary tumor samples, one SCLC and one adenocarcinoma (Table 1). High-level amplification on chromosome 12q13-12q14, encompassing the CDK4 locus, was found in two samples (Table 1; FIG. 6D). High-level amplification of this region has been reported in several different tumor types, including bladder carcinoma (53), glioma (39), osteosarcoma (54) and lung cancer (40). The amplified region contains from 20 to 34 genes, among which CDK4 is an intriguing candidate oncogene (Table 5). Amplification of CDK4 in the two samples and three others with lower-level copy number gain was verified by quantitative real-time PCR (Table 1). Two to three-fold amplification of CDK6 was also found in four adenocarcinoma samples (Table 5).

### Example 7: Tyrosine Kinase Copy Number Alterations

A survey of tyrosine kinase gene copy number identified four receptor tyrosine kinase genes (RTKs), FGFR1, ERBB2 (Her-2/neu), MET (HGFR), and EGFR, as being highly amplified (copy number >=8) in at least one sample. Three additional tyrosine kinases had a copy number of at least 6 in one or more samples and a total of six tyrosine kinase genes showed a copy number of 4 or more in at least 5 samples. These kinase genes were found in regions containing several genes and therefore the targets of the amplicons are still unknown.

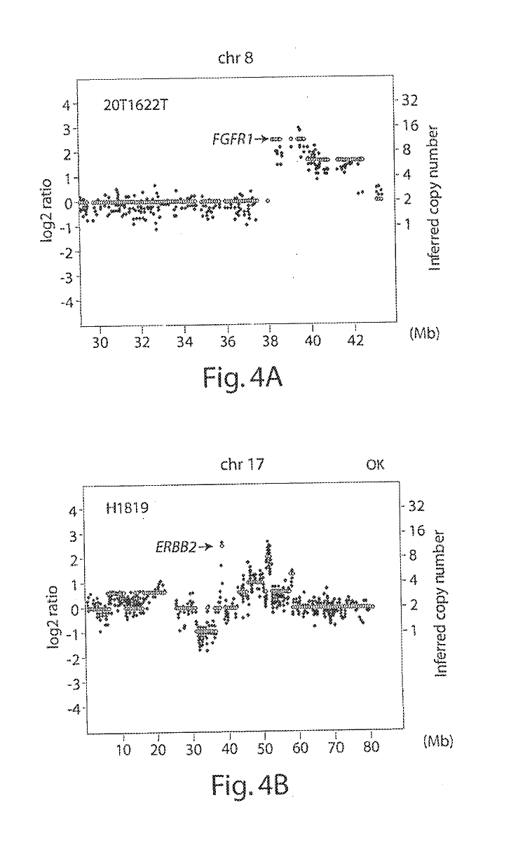

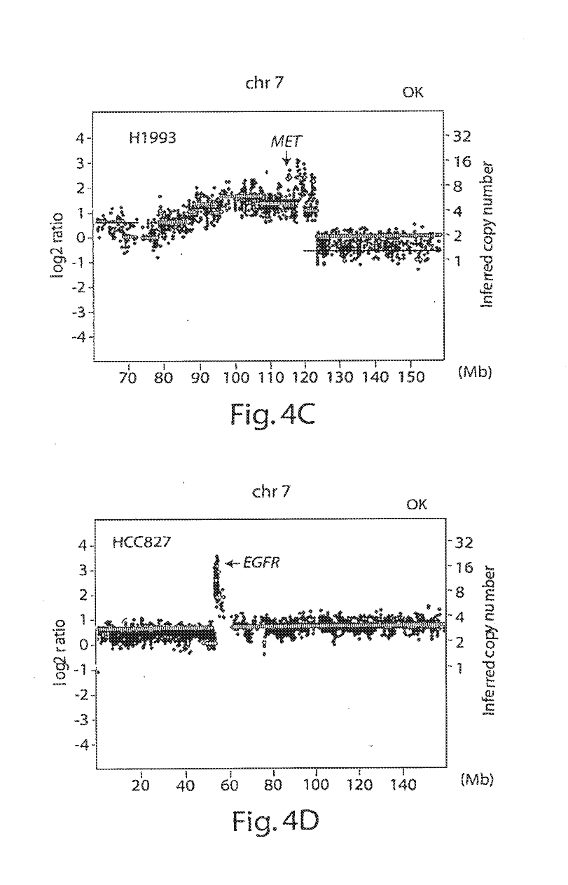

A novel amplification of chromosome 8p11.2-p11.1 was identified, which includes the FGFR1 gene. High-level amplification (copy number >=8) was found in two NSCLC samples, S0449T and MGH1622T (FIG. 4A) and was confirmed by real time PCR (Table 1); lower level copy number gains (copy number >=4) were found in four additional NSCLC samples. Amplification of FGFR1 has been found in other tumor types, including breast and urinary carcinoma (42, 43, 55). However, the role of FGFR1 in lung tumorigenesis remains largely unknown and there is not yet any evidence to implicate FGFR1 as the target of this amplicon.

The identification of ERBB2, an RTK shown to be overexpressed in many human tumors, including breast, colorectal, ovarian and non-small cell lung cancers (56), has led to the development of the targeted breast cancer therapy, trastuzumab (Herceptin) (57). Over four-fold amplification (copy number >=8) of the region surrounding ERBB2 was found in one adenocarcinoma cell line, H1819 (FIG. 4B). Additional primary tumors, including two adenocarcinoma samples and one SCLC sample, had moderate copy number gains (copy number >=4) of the ERBB2 locus. Overexpression and activating mutations of another RTK, MET, have been found in a variety of tumor types (56). A high-level amplification (copy number >9) of chromosome 7q31.2 resolved into 3 peaks in the H1993 sample. One of these peaks was a 390 kb amplicon which included MET (FIG. 4C); lower-level gain of this region was found in two additional NSCLC samples.

### Example 8: EGFR Mutation Compared to EGFR Amplification

The amplification of the EGFR region was analyzed in an unbiased fashion and to determine the degree or correlation between amplification, mutation (12) and expression (58) of EGFR within the same lung carcinoma samples. This analysis revealed amplification of the EGFR region on chromosome 7p11.2 to copy number >8 in one primary squamous cell carcinoma (S0480T), one adenocarcinoma cell line (HCC827) (FIG. 4D) and one primary adenocarcinoma (AD347T) (Table 1 and Table 2). Interestingly, HCC827 also contains an EGFR mutation (Table 2), while AD347T and S0480T do not.

Comparisons of EGFR amplification, mutation and expression data indicate that high and moderate level amplifications are not always associated with EGFR gene mutation (Table 2). In addition, seventeen samples run on SNP arrays, with known EGFR mutation status, also had expression information available (Table 2). Samples with EGFR mutation and/or copy number gain (copy number >=4) of the EGFR gene were shown to have higher average EGFR expression than samples with wildtype, unamplified EGFR on average, as measured by 2 probe sets on Affymetrix U95AV2 arrays (1537_at (EGFR): p=0.035, 37327_at (EGFR): p=0.004). This study shows that mutations in the EGFR gene are at least partially independent of EGFR gene amplification and that EGFR expression and amplification are correlated.

TABLE 1
Predicted regions of recurrent amplification^{a} and homozygous deletion^{b}

| | | | | | | | Real- | | |
|---|---|---|---|---|---|---|---|---|---|
| | | | | | Mean | | time | | |
| | | | | | dCHIP | Binary | PCR | Representative | Total |
| | Start | Stop | Size | | copy | segmentation | copy | gene within | # of |
| Cytoband^{c} | (Mb)^{c} | (Mb)^{c} | (Mb) | Sample | number | copy number | number^{ce} | interval^{ef} | genes^{c} |
| 1p34.2 | 39.51 | 40.55 | 1.04 | H1963 | 10.6 | 11 | 147.80 | MYCL1 | 23 |
| | 39.55 | 40.91 | 1.36 | S0173T | 10.7 | 8 | 20.70 | | 24 |
| 1q31.1 | 184.74 | 185.02 | 0.28 | H524 | 0 | | | | 0 |
| | 184.76 | 185.01 | 0.25 | S0187T | 0 | | | | 0 |
| 1q31.3 | 193.15 | 193.89 | 0.74 | S0171T | 0 | | | KCNT2 | 1 |
| | 193.03 | 193.96 | 0.93 | H1819 | 0 | | | | 1 |
| 2p24.3-p24.2 | 14.20 | 16.38 | 2.18 | S0172T | 14.2 | 12 | 56.20 | MYCN | 9 |
| | 15.25 | 17.06 | 1.81 | H526 | 7.0 | 5 | 42.30 | | 9 |
| 2q22.1 | 141.71 | 142.45 | 0.73 | H2122 | 0 | 0 | 0.01 | LRPIB | 1 |
| | 141.79 | 142.78 | 0.99 | HCC95 | 0 | 1 | 0.00 | | 1 |
| | 141.94 | 142.20 | 0.26 | H2126 | 0 | 0 | 0.00 | | 1 |
| | 142.00 | 142.20 | 0.20 | H157 | 0 | 0 | 0.06 | | 1 |
| | 141.99 | 142.21 | 0.22 | H1339 | 0 | | | | 1 |
| 3p14.2 | 60.29 | 60.78 | 0.49 | HCC95 | 0 | 0 | | FHIT | 4 |
| | 60.32 | 60.40 | 0.08 | H2887 | 0 | 0 | 0.00 | | 1 |
| 3q25.1 | 152.82 | 152.95 | 0.12 | H2882 | 0 | 0 | 0.00* | AADAC, SUCNR1 | 2 |
| | 152.82 | 152.95 | 0.12 | S0177T | 0 | 0 | 0.02* | | 2 |
| 3q26.31-q27.1 | 174.86 | 184.52 | 9.66 | S0465T | 7.8 | 5 | 10.29 | PIK3CA | 41 |
| | 182.50 | 184.47 | 1.98 | S0515T | 13.2 | 11 | 3.90 | | 12 |
| 4q22.1 | 92.20 | 92.57 | 0.37 | H2126 | 0 | | | TMSL3 | 1 |
| | 92.12 | 92.51 | 0.39 | H1975 | 0 | | | | 1 |
| 4q34.3 | 182.84 | 183.18 | 0.34 | H2087 | 0 | | | | 0 |
| | 182.98 | 183.52 | 0.54 | S0169T | 0 | | | | 0 |
| 7p12.1-q11.22 | 53.16 | 61.49 | 11.34 | HCC827 | 11.3 | 9 | 41.66 | EGFR | 50 |
| | 54.24 | 69.62 | 9.73 | AD347T | 9.8 | 9 | 18.30 | | 123 |
| | 54.37 | 55.63 | 13.65 | S0480T | 13.7 | 10 | 47.84 | | 12 |
| 8p12-p11.22 | 38.05 | 39.97 | 1.93 | MGH1622T | 10.4 | 11 | 14.92 | FGFR1 | 22 |
| | 38.73 | 39.84 | 1.11 | S0449T | 6.4 | 5 | 6.07 | | 11 |
| 8p23.3 | 0.18 | 2.57 | 2.39 | H2009 | 0 | | | | 0 |
| | 0.18 | 0.80 | 0.61 | AD186T | 0 | | | | 0 |
| | 0.23 | 0.80 | 0.57 | S0506T | 0 | | | | 0 |
| 8p23.1 | 9.45 | 10.15 | 0.70 | HCC1171 | 0 | | | TNKS, MSRA | 2 |
| | 9.73 | 10.05 | 0.32 | S0530T | 0 | | | | 2 |
| 8q24.13-q24.21 | 126.60 | 128.89 | 2.30 | H524 | 6.6 | 5 | 174.51 | MYC | 6 |
| | 127.46 | 128.89 | 1.43 | HCC827 | 6.9 | 6 | 8.63 | | 6 |
| | 127.59 | 130.83 | 3.24 | NCI-H23 | 8.0 | 7 | 11.11 | | 11 |
| | 127.90 | 129.62 | 1.72 | H2122 | 3.6 | 5 | 14.49 | | 6 |
| | 128.44 | 129.60 | 1.16 | H2087 | 7.9 | 8 | 15.99 | | 4 |
| 9p23 | 8.61 | 9.12 | 0.51 | S077T | 0 | 0 | 0.01 | PTPRD | 2 |
| | 8.79 | 9.55 | 0.77 | H358 | 0 | 0 | 0.00* | | 2 |
| | 9.41 | 9.61 | 0.20 | HCC1171 | 0 | 0 | 0.08* | | 1 |
| | 9.50 | 9.75 | 0.25 | H2347 | 0 | 0 | 0.00* | | 1 |
| | 7.50 | 7.51 | 0.01 | H1339 | 0 | | | | 2 |
| 9p21.3 | 20.90 | 22.94 | 2.03 | H2126 | 0 | 0 | 0.00 | CDKN2A | 35 |
| | 21.20 | 22.19 | 0.98 | HCC1359 | 0 | 0 | 0.01 | | 21 |
| | 21.58 | 25.10 | 3.52 | HCC1171 | 0 | 0 | 0.00 | | 11 |
| | 21.70 | 23.39 | 1.69 | H2882 | 0 | 1 | 0.00 | | 6 |
| | 21.84 | 26.83 | 4.99 | HCC95 | 0 | 0 | 0.00 | | 11 |
| | 21.95 | 22.09 | 0.14 | H2122 | 0 | 0 | 0.01 | | 3 |
| | 24.34 | 24.70 | 0.36 | H157 | 0 | 0 | 0.03* | | 1 |
| | 21.18 | 24.22 | 3.04 | S0169T | 0 | | | | 18 |
| | 21.54 | 22.07 | 0.53 | NCI.H226 | 0 | | | | 3 |
| | 21.64 | 24.90 | 3.26 | S0530T | 0 | | | | 5 |
| | 21.95 | 24.10 | 2.15 | H1650 | 0 | | | | 2 |
| | 24.52 | 24.69 | 0.17 | H1339 | 0 | | | | 1 |
| 10q23.31 | 89.03 | 89.40 | 0.37 | H1607 | 0 | 0 | 0.00 | PTEN | 4 |
| | 89.18 | 89.88 | 0.69 | S0187T | 0 | 0 | 0.09 | | 4 |
| | 89.35 | 91.16 | 1.80 | S0189T | 0 | 0 | 0.12* | | 22 |
| 12p11.21 | 32.17 | 33.02 | 0.85 | S0515T | 8.8 | 7 | 10.75 | PKP2 | 6 |
| | 32.69 | 36.59 | 3.90 | H2087 | 7.8 | 8 | 11.43 | | 12 |
| 12q13.3-q14.1 | 56.26 | 57.37 | 1.10 | H2087 | 8.8 | 9 | 23.40 | CDK4 | 20 |
| | 55.82 | 56.67 | 0.85 | HCC827g | 13.0 | 13 | 30.34 | | 34 |
| 13q14.2 | 46.72 | 47.51 | 0.79 | H2009 | 0 | | | RBI | 4 |
| | 46.97 | 47.24 | 0.27 | H378 | 0 | | | | 1 |
| 19q12 | 34.02 | 35.55 | 1.53 | S0524T | 6.7 | 7 | 6.77 | CCNE1 | 12 |
| | 34.79 | 37.09 | 2.30 | S0188T | 7.9 | 7 | 10.93 | | 12 |
| 22q11.21-q11.22 | 16.99 | 20.31 | 3.32 | H1819 | 6.8 | 7 | 12.57 | CRKL | 92 |
| | 17.51 | 21.44 | 3.93 | HCC515 | 7.4 | 7 | 14.01 | | 169 |
| | 18.47 | 20.61 | 2.14 | S0380T | 6.4 | 5 | 8.44 | | 68 |
| | 19.45 | 20.75 | 1.29 | HCC1359 | 6.5 | 7 | 8.05 | | 48 |

^{a}Predicted regions containing at least 4 SNPs, at least 5 kb in size and with an inferred copy number of >=7, which occur in 2 or more samples; Amplified regions separated by <2 Mb of unamplified sequenced have been combined
^{b}Predicted regions of at least 5 kb in size containing at least 4 consecutive SNPs with inferred copy number = 0, which occur in 2 or more samples; Deleted regions separated by <2 Mb of undeleted sequence have been combined
^{c}Based on hg16 genome assembly
^{d}Mean of amplified segments include sequences with copy number <7, if regions were combined
^{e}Real-time PCR values marked by an * denotes those targets that are not in an exon of the representative gene
^{f}Bold indicates only gene in region
^{g}Less than 4 SNPs, but validated with real-time PCR

TABLE 2
Comparison of EGFR mutation, amplification and expression^{a}

| | | | EGFR copy | | EGFR | EGFR |
|---|---|---|---|---|---|---|
| | | SNP | number | EGFR kinase domain | expression | expression |
| Sample | Histology | Dataset^{b} | (Dchip) | mutation^{c} | 1537_at^{d} | 37327_at d |
| S0480T | Squamous | 120K | 14 | none | N/A | N/A |
| HCC827 | Adenocarcinoma | 120K | 14 | E746_A750del | N/A | N/A |
| AD347T | Adenocarcinoma | 120K | 11 | none | 1534.43 | 699.58 |
| H3255 | Adenocarcinoma | 60K | 8 | L858R | 4059.73 | 1913.25 |
| H1819 | Adenocarcinoma | 120K | 4 | N/A | N/A | N/A |
| H1993 | Adenocarcinoma | 120K | 4 | N/A | N/A | N/A |
| S0405T | Adenocarcinoma | 120K | 4 | E746_A750del | N/A | N/A |
| DFCI-LU-01 | Adenocarcinoma | 60K | 4 | L747_E749del, A750P | N/A | N/A |
| S0412T | Adenocarcinoma | 120K | 3 | E746_A750del | N/A | N/A |
| S0514T | Adenocarcinoma | 120K | 3 | G719S | N/A | N/A |
| S0380T | Adenocarcinoma | 120K | 3 | E746_A750del | N/A | N/A |
| AD309T | BAC | 120K | 3 | L747_S752del, P753S | 298.48 | 31.54 |
| AD157 | Adenocarcinoma | 120K | 3 | none | 119.39 | 104.56 |
| AD337 | Adenocarcinoma | 120K | 3 | none | 338.97 | 221.75 |
| H1975 | Adenocarcinoma | 120K | 3 | L858R | N/A | N/A |
| S0377T | Adenocarcinoma | 120K | 2 | G719S | N/A | N/A |
| H358 | BAC | 120K | 2 | none | 183.33 | 75.11 |
| AD327 | Adenocarcinoma | 120K | 2 | none | 94.58 | 43.09 |
| AD338 | Adenocarcinoma | 120K | 2 | none | 127.80 | 39.51 |
| AD311 | Adenocarcinoma | 120K | 2 | none | 33.51 | 35.80 |
| AD362 | Adenocarcinoma | 120K | 2 | none | 48.22 | 36.63 |
| AD330 | Adenocarcinoma | 120K | 2 | none | 44.88 | 14.70 |
| AD334 | Adenocarcinoma | 120K | 2 | none | 31.65 | 63.74 |
| AD335 | Adenocarcinoma | 120K | 2 | none | 24.27 | 35.41 |
| AD336 | Adenocarcinoma | 120K | 2 | none | 132.19 | 82.49 |
| AD163 | Adenocarcinoma | 120K | 2 | none | 29.66 | 33.82 |
| H1650 | Adenocarcinoma | 60K | 2 | E746_A750del | 2125.20 | 640.89 |
| H1666 | Adenocarcinoma | 60K | 2 | none | 600.01 | 205.29 |

N/A = not available
^{a}Samples with EGFR amplification (cn >=4), or EGFR gene mutation; or those with EGFR expression, EGFR copy number and EGFR mutation status available
^{b}120K set was from both XbaI and HindIII chips, 60K set was from only XbaI chip
^{c}(Paez et al., 2004; Naoki et al., submitted)
^{d}(Bhattacharjee at al., 2001; Naoki et al., submitted; unpublished data)

TABLE 3
All predicted homozygous deletions^{a}

| Chromosome^{b} | Start (MB)^{b} | Stop (Mb)^{b} | Sample |
|---|---|---|---|
| 1 | 184.74 | 185.02 | H524 |
| 1 | 184.76 | 185.01 | S0187T |
| 1 | 193.03 | 193.96 | H1819 |
| 1 | 193.15 | 193.89 | S0171T |
| 2 | 18.36 | 22.20 | H2882 |
| 2 | 51.02 | 51.59 | S0177T |
| 2 | 141.71 | 142.45 | H2122 |
| 2 | 141.79 | 141.88 | HCC95 |
| 2 | 141.94 | 142.20 | H2126 |
| 2 | 142.00 | 142.20 | H157 |
| 2 | 142.21 | 142.57 | HCC95 |
| 2 | 142.70 | 142.78 | HCC95 |
| 2 | 141.99 | 142.21 | H1339 |
| 3 | 0.05 | 2.37 | S0196T |
| 3 | 49.85 | 50.82 | S0189T |
| 3 | 60.29 | 60.78 | HCC95 |
| 3 | 60.32 | 60.40 | H2887 |
| 3 | 62.24 | 62.57 | S0170T |
| 3 | 76.73 | 77.22 | HCC95 |
| 3 | 85.27 | 85.79 | H1963 |
| 3 | 152.82 | 152.95 | H2882 |
| 3 | 152.82 | 152.95 | S0177T |
| 4 | 38.18 | 38.31 | S0194T |
| 4 | 92.20 | 92.57 | H2126 |
| 4 | 92.12 | 92.51 | H1975 |
| 4 | 131.18 | 131.30 | H524 |
| 4 | 169.50 | 169.72 | S0188T |
| 4 | 182.98 | 183.52 | S0169T |
| 4 | 182.84 | 183.18 | H2087 |
| 5 | 55.22 | 55.44 | H1607 |
| 5 | 102.93 | 103.03 | H2126 |
| 5 | 114.65 | 115.10 | S0189T |
| 5 | 136.38 | 136.49 | H1963 |
| 6 | 53.54 | 54.11 | H1993 |
| 6 | 162.84 | 163.19 | HCC515 |
| 7 | 29.17 | 29.30 | H1607 |
| 8 | 0.18 | 2.57 | H2009 |
| 8 | 0.18 | 0.80 | AD186T |
| 8 | 0.23 | 0.80 | S0506T |
| 8 | 9.45 | 10.15 | HCC1771 |
| 8 | 9.73 | 10.05 | S0530T |
| 8 | 14.17 | 14.28 | S0187T |
| 8 | 16.42 | 16.54 | H2887 |
| 8 | 137.65 | 137.86 | H2122 |
| 9 | 5.42 | 5.84 | H1993 |
| 9 | 7.50 | 7.51 | H1339 |
| 9 | 8.61 | 9.12 | S0177T |
| 9 | 8.79 | 8.92 | H358 |
| 9 | 9.41 | 9.55 | H358 |
| 9 | 9.41 | 9.61 | HCC1771 |
| 9 | 9.50 | 9.75 | H2347 |
| 9 | 10.03 | 10.07 | S0458T |
| 9 | 20.90 | 22.94 | H2126 |
| 9 | 21.20 | 22.19 | HCC1359 |
| 9 | 21.58 | 25.10 | HCC1771 |
| 9 | 21.70 | 22.94 | H2882 |
| 9 | 21.84 | 26.83 | HCC95 |
| 9 | 21.95 | 22.09 | H2122 |
| 9 | 21.18 | 24.22 | S0169T |
| 9 | 21.54 | 22.07 | NCI.H226 |
| 9 | 21.64 | 24.90 | S0530T |
| 9 | 21.95 | 22.10 | H1650 |
| 9 | 24.52 | 24.69 | H1339 |
| 9 | 23.15 | 23.39 | H2882 |
| 9 | 24.34 | 24.70 | H157 |
| 10 | 11.23 | 11.80 | H2126 |
| 10 | 34.63 | 34.79 | H157 |
| 10 | 52.11 | 53.33 | H1993 |
| 10 | 55.79 | 56.19 | S0169T |
| 10 | 68.10 | 68.59 | H1963 |
| 10 | 75.49 | 76.13 | H1963 |
| 10 | 89.03 | 89.40 | H1607 |
| 10 | 89.18 | 89.88 | S0187T |
| 10 | 89.35 | 91.16 | S0189T |
| 10 | 116.49 | 117.43 | S0189T |
| 12 | 3.46 | 3.89 | H1993 |
| 13 | 46.72 | 47.51 | H2009 |
| 13 | 46.97 | 47.24 | H378 |
| 13 | 51.45 | 54.41 | H2009 |
| 13 | 54.57 | 55.11 | S0177T |
| 17 | 12.16 | 12.40 | S0187T |
| 17 | 13.55 | 13.66 | H2347 |
| 17 | 20.80 | 22.27 | S0187T |
| 18 | 19.83 | 24.48 | H2887 |
| 18 | 26.34 | 26.72 | NCI.H226 |
| 18 | 69.09 | 69.88 | HCC461 |
| 21 | 9.93 | 24.51 | HCC366 |
| 23 | 6.43 | 7.24 | H157 |
| 23 | 25.29 | 26.27 | S0187T |
| 23 | 31.11 | 31.53 | S0412T |
| 23 | 126.71 | 127.03 | H1607 |

^{a}Predicted regions of at least 5 kb in size containing at least 4 consecutive SCPs with inferred copy number = 0
^{b}Based on hg 16 genomes assembly

TABLE 4
All predicted high-level amplifications^{a}

| | | | | Mean dCHIP |
|---|---|---|---|---|
| Chromosome^{b} | Start (Mb)^{b} | Stop (Mb)^{b} | Sample | copy number |
| 1 | 30.81 | 31.36 | H526 | 5.3 |
| 1 | 34.87 | 35.29 | H2122 | 5.3 |
| 1 | 39.51 | 40.55 | H1963 | 10.6 |
| 1 | 39.55 | 40.91 | S0173T | 10.7 |
| 1 | 56.91 | 65.68 | S0198T | 9.2 |
| 1 | 110.98 | 117.67 | S0173T | 7.6 |
| 1 | 242.77 | 243.19 | S0377T | 5.3 |
| 2 | 14.20 | 16.38 | S0172T | 14.2 |
| 2 | 15.25 | 17.06 | H526 | 7.0 |
| 3 | 169.50 | 170.89 | S0465T | 6.9 |
| 3 | 174.86 | 175.18 | S0465T | 8.6 |
| 3 | 177.17 | 184.52 | S0465T | 7.0 |
| 3 | 182.50 | 184.47 | S0515T | 13.2 |
| 4 | 44.57 | 45.85 | S0380T | 7.9 |
| 5 | 8.88 | 14.31 | S0376T | 7.1 |
| 5 | 38.45 | 41.72 | S0188T | 7.0 |
| 5 | 90.09 | 90.65 | MGH1622T | 6.7 |
| 5 | 98.13 | 100.62 | MGH1622T | 4.8 |
| 5 | 104.61 | 104.81 | MGH1622T | 5.9 |
| 5 | 157.83 | 157.95 | MGH1622T | 6.13 |
| 6 | 11.60 | 11.96 | HCC827 | 15.6 |
| 6 | 84.24 | 85.58 | S0480T | 7.9 |
| 6 | 135.41 | 135.58 | H526 | 10.3 |
| 7 | 53.16 | 61.49 | HCC827 | 11.3 |
| 7 | 54.24 | 69.62 | AD347T | 9.7 |
| 7 | 54.37 | 55.63 | S0480T | 13.7 |
| 7 | 115.70 | 116.09 | H1993 | 9.1 |
| 7 | 118.07 | 123.35 | H1993 | 7.1 |
| 8 | 32.16 | 32.30 | HCC95 | 7.8 |
| 8 | 36.33 | 36.48 | S0449T | 7.3 |
| 8 | 38.05 | 39.97 | MGH1622T | 10.4 |
| 8 | 38.73 | 39.84 | S0449T | 6.4 |
| 8 | 40.30 | 42.23 | S0458T | 9.2 |
| 8 | 61.86 | 62.62 | S0177T | 13.6 |
| 8 | 74.22 | 76.27 | HCC827 | 6.9 |
| 8 | 80.79 | 82.81 | HCC827 | 6.9 |
| 8 | 100.57 | 100.75 | AD309T | 6.7 |
| 8 | 103.18 | 104.22 | HCC827 | 6.9 |
| 8 | 124.15 | 124.52 | HCC827 | 6.3 |
| 8 | 126.60 | 128.89 | H524 | 6.6 |
| 8 | 127.46 | 128.89 | HCC827 | 6.9 |
| 8 | 127.59 | 130.83 | NCI.H23 | 8.0 |
| 8 | 127.90 | 129.62 | H2122 | 3.6 |
| 8 | 128.44 | 129.60 | H2087 | 7.9 |
| 8 | 133.08 | 133.65 | HCC827 | 6.8 |
| 9 | 13.09 | 13.46 | H1607 | 6.6 |
| 9 | 27.15 | 27.32 | S0515T | 6.7 |
| 9 | 39.01 | 62.64 | S0465T | 6.4 |
| 9 | 98.96 | 99.02 | S0465T | 6.3 |
| 10 | 86.63 | 87.16 | HCC1359 | 6.8 |
| 11 | 34.05 | 39.37 | HCC95 | 9.8 |
| 11 | 48.21 | 51.30 | HCC95 | 6.8 |
| 11 | 65.15 | 69.34 | HCC515 | 6.7 |
| 11 | 103.48 | 104.36 | H1993 | 7.9 |
| 12 | 32.17 | 33.02 | S0515T | 8.8 |
| 12 | 32.69 | 36.59 | H2087 | 7.8 |
| 12 | 56.26 | 57.37 | H2087 | 8.8 |
| 12 | 59.44 | 59.78 | H2087 | 7.1 |
| 12 | 62.95 | 63.61 | HCC827 | 9.5 |
| 12 | 66.50 | 68.51 | S0372T | 7.0 |
| 14 | 27.57 | 27.87 | S0480T | 7.4 |
| 14 | 32.90 | 35.11 | H1819 | 7.0 |
| 15 | 43.79 | 44.74 | AD334T | 6.4 |
| 15 | 48.27 | 51.62 | AD334T | 7.4 |
| 17 | 22.27 | 25.81 | H2887 | 6.0 |
| 17 | 35.25 | 37.30 | HCC515 | 10.5 |
| 17 | 37.93 | 39.11 | H1819 | 8.0 |
| 17 | 51.10 | 52.51 | H1819 | 7.9 |
| 19 | 34.02 | 35.55 | S0524T | 6.7 |
| 19 | 34.79 | 37.09 | S0188T | 7.9 |
| 19 | 43.01 | 45.00 | S0515T | 6.0 |
| 20 | 49.02 | 49.81 | AD334T | 6.8 |
| 20 | 52.72 | 53.40 | AD334T | 6.5 |
| 21 | 17.51 | 18.48 | HCC827 | 6.9 |
| 22 | 16.99 | 20.31 | H1819 | 6.8 |
| 22 | 17.51 | 21.44 | HCC515 | 7.4 |
| 22 | 18.47 | 20.61 | S0380T | 6.4 |
| 22 | 19.45 | 20.75 | HCC1359 | 6.5 |
| 22 | 27.84 | 30.18 | S0480T | 6.6 |

^{a}Predicted regions containing at least 4 SNPa, at least 5 kb in size and with an inferred copy number of > or = 7
^{b}Based on hg 16 genome assembly

TABLE 5
Genes within selected regions of amplification

| | Minimally | | | Samples with copy | |
|---|---|---|---|---|---|
| | overlapped | | Samples with | number >=4 and | New samples |
| Cytobandb | region^{b,c} | Candidate genes^{d} | copy number >=7 | <7 | with amplification |
| 1p34.2 | 39.51-40.55 | HEY1, NTSC1A, HPCAL4, PPIE, OXCT2, | H1963, S0173T | H1184, S0187T, | AD375 (4 to 7), |
| | | BMP8B, TRIT1, MYCL1, FLJ14490, CAP1, PPT1, | | S0193T, S0480T | H220 (4 to 7), |
| | | LOC441883, RLF, LOC127391, ZMPSTE24, | | | HCC33 (>7), H1450 |
| | | COL9A2, LOC388621, LOC64744, LOC65243, | | | (4 to 7), H378 (4 to |
| | | FLJ16030, FLJ21144, MGC27466, RIMS3 | | | 7) |
| 2p24.3 | 16.00-16.38 | MYCNOS, MYCN, LOC391353 | S0172T, H526 | H1437, S0190T | |
| 3q26.31 | 179.21-180.44 | KCNMB2, WIG1, PIK3CA, KCNMB3, ZNF639, | S0465T | S0466T, H1819, | KP54_H10 (4 to 7) |
| | | MFN1, GNB4 | | HCC95, | KP53_E5 (>7), |
| | | | | S0168T, S0169T, | KP53_D9 (4 to 7), |
| | | | | S0187T, | KP54_A12 (4 to 7) |
| | | | | MGH1622T, | |
| | | | | H2882, S0515T | |
| 3q26.31 | 183.75-184.35 | ATP11B, RP42, MCCC1, LAMP3, RNU3P4, | S0465T, S0515T | S0446T, H1819, | KP54_H10 (4 to 7) |
| | | KIAA0861, B3GNT5 | | HCC95, S0168T, | KP53_E5 (>7), |
| | | | | S0169T, S0173T, | KP53_D9 (4 to 7), |
| | | | | S0187T, | KP54_A12 (4 to 7) |
| | | | | MGH1622T | |
| 7p12.1-p11.2 | 54.36-54.84 | MGC335300, LOC392030, SEC61G, LOC441226, | HCC827, AD347T, | S0405T, H1819, | KP54_D5 (>7) |
| | | EGFR | SG480T | H1193 | KP53_F11 (>7), |
| | | | | | H3255 (>7) |
| 7q21.2 | 90.81-92.22 | MTERF, AKAP9, CYP51A1, LOC401387, CCM1, | | S0464T, H1993, | |
| | | ANK1B1, LOC402286, ODAG, ERVWEI, PEX1, | | H2122, AD311T | |
| | | DKFZ564O0523, LOC442333, MGC40405, | | | |
| | | CDK6, LOC441267, RN7SLP4 | | | |
| 7q31.2 | 115.7-117.42 | CAV2, CAV1, MET, CAPZA2, ST7, WNT2, | H1993 | H2009, HOP-92 | |
| | | ASZ1, CFTR, CTTNBPS, ISM8, ANKRD7 | | | |
| 8p11.23 | 38.05-39.21 | LSM1, BAG4, DDHD2, HTPAP, WHSC1L1, | S0449T, | S0480T, H2882, | KP53_A3 (4 to 7), |
| | | LOC441345, LETM2, FGFR1, FLJ43582, | MGH1622T | HCC1359, HCC95 | H1703 (4 to 7), |
| | | LOC286140, TACC1, PLEKHA2, FLJ90724, | | | S0446 (4 to 7), |
| | | BLP1, ADAM9, ADAM32, ADAM5 | | | KP54_E8, (4 to 7), |
| | | | | | KP53_E10 (4 to 7), |
| | | | | | KP54_E7 (4 to 7), |
| 8p11.23 | 39.81-41.95 | SFRP1, SLDS, MYST3, ANK1, GOLGA7 | | | KP54_F11 (4 to 7), |
| | | | | | KP54_B8, (4 to 7), |
| | | | | | KP53_A3 (4 to 7), |
| | | | | | KP53_E10, (4 to 7), |
| | | | | | S0458T (>7), |
| | | | | | KP54_E7 (4 to 7), |
| | | | | | MGH1622T (4 to 7) |
| 8q12.2-q12.3 | 61.86-62.22 | LOC442389, NASPP1, LOC157813, LOC441350, | S0177T | S0170T, H1993, | |
| | | (MGC34646, ASPH) | | H2882, HCC827 | |
| 8q24.21 | 128.66-128.89 | MYC, PVT1 | H2122, H524, | S0535T, H2882, | KP54_E4 (>7), |
| | | | NCI-H23, H2087, | H2887, HCC827, | AD228T (4 to 7), |
| | | | HCC827 | S0170T, S0194T, | H1975 (>7), |
| | | | | AD330T, H358 | KP54_D5 (4 to 7) |
| 11p13 | 34.69-37.65 | CD44, FJX1, TRIM44, RAG1, TRAF6 | | | KP54_B8 (4 to 7), |
| | | | | | HCC95 (>7), |
| | | | | | S0496T (4 to 7), |
| | | | | | S0464T (4 to 7) |
| 11q13.3 | 68.58-69.34 | TPCN2, MYEOV, LOC390218, LOC399919, | HCC515 | HCC366, HOP-62, | KP54_E4 (4 to 7), |
| | | LOC44049, ORAOV1, FGF19, FGF4, FGF3, | | NCI-H23, HCC78 | KP54_B11 (4 to 7), |
| | | CCND1 | | | S0506T (4 to 7), |
| 12p11.21 | 32.69-33.21 | DNM11, CGI-04, PKP2, LOC283343 | S0515T, H2087 | S0372T, S0376T, | HCC33 (4 to 7), |
| | | | | H157, H1993, | H1339 (4 to 7), |
| | | | | H2009. HCC461, | H1703 (4 to 7), |
| | | | | HOP-2 | KP54_H10 (4 to 7) |
| 12q13.3 | 56.26-56.15 | KIF5A, PIP5K2C, DTX3, GEFT, SLC26A10, | H2087, HCC827 | H1993, S0372T, | AD350T (4 to 7) |
| | | GALGT, LOC441641, OS-9, CENTG1, SAS, | | S0514T, S0539T | |
| | | CDK4, LOC92979, CYP27B1, METTL1, | | | |
| | | DKFZP586D0919, TSFM, AVIL, CTDSP2 | | | |
| 19q12 | 34.79-35.55 | POP4, PLEKHF1, C19orf2, LOC126170, | S0188T, S0527T | H2887 | KP53_D9 (4 to 7), |
| | | C19orf12, TAF2GI, CCNE1 | | | KP54_C12 (4 to 7), |
| | | | | | KP54_E4 (4 to 7), |
| 22q11.21 | 19.45-20.31 | PIK4CA, SERPIND1, SNAP29, CRKI, | HCC515, H1819, | S0464T, S0446T | S0528T (4 to 7) |
| | | LOC400890, LOC400890, FLJ30473, FLJ30473, | HCC1359, S0380T | H524 | |
| | | LZTR1, THAP7, LOC439931, MGC16703, | | | |
| | | P2RXL1, SLC7A4, LOC440799, LOC400891, | | | |
| | | LOC402037, FLJ42953, LOC440800, | | | |
| | | LOC376818, LOC284861, LOC440801, | | | |
| | | LOC440802, LOC400888, LOC388846, | | | |
| | | LOC440803, LOC440803, LOC391303, | | | |
| | | LOC391298, LOC388853, LOC440804, HIC2, | | | |
| | | LOC150221, UBE2L3, LOC150223 | | | |

^{a}Regions with at least two amplification =>7 or regions surrounding other loci discussed in text
^{b}Based on hg16 assembly
^{c}Region of minimal overlap between samples, of copy number >=4
^{d}Bold indicates likely oncogene; genes in parantheses occur outside of minimal region, but within the high-level amplicon of at least one sample

### EQUIVALENTS

Those skilled in the art will recognize, or be able to ascertain using no more than routine experimentation, numerous equivalents to the specific procedures described herein. Such equivalents are considered to be within the scope of the invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the invention and embodiments thereof.

What is claimed is:

1. A method of diagnosing cancer or a predisposition thereto in a subject, comprising
a. providing a biological sample from the subject; and
b. determining in the biological sample the copy number of a one or more nucleic acids selected from the group consisting of:
i. an ASPH gene or fragment thereof;
ii. a region of human chromosome 8q12.1-q13.11;
iii. a MGC24646 gene or fragment thereof;
iv. a region of human chromosome 12p11;
v. a LOC283343 or fragment thereof;
vi. a CGI-04 gene or fragment thereof;
vii. a DNM1L gene or fragment thereof;
viii. a PKP2 gene or fragment thereof;
ix. a region of human chromosome 22q11;
x. a CRKL gene or fragment thereof; and
xi. a PIK4CA gene or fragment thereof;
wherein a copy number greater than two of said nucleic acid indicates that the subject has cancer or a predisposition thereto.

2. The method of claim 1, wherein said cancer is lung cancer.

3. The method of claim 2, wherein said lung cancer is small cell lung cancer, lung adenocarcinoma or large cell carcinoma.

4. The method of claim 1, wherein said copy number is greater than four.

5. The method of claim 1, wherein said copy number is greater than ten.

6. The method of claim 1, wherein said copy number is greater than twenty.

7. The method of claim 1, wherein said copy number is greater than forty.

8. The method of claim 1, wherein said nucleic acid is greater than about 50 kilobases in size.

9. The method of claim 1, wherein said nucleic acid is greater than about 100 kilobases in size.

10. The method of claim 1, wherein said nucleic acid is greater than about 500 kilobases in size.

11. The method of claim 1, wherein said nucleic acid is greater than about 670 kilobases in size.

12. The method of claim 1, wherein said copy number is determined by a method selected from the group consisting of real time polymerase chain reaction, single nucleotide polymorphism (SNP) arrays, and interphase fluorescent in situ hybridization (FISH) analysis.

13. A method of diagnosing cancer or a predisposition thereto in a subject, comprising:
a. providing a biological sample from the subject; and
b. determining in the biological sample the presence of a one or more deletions on
i. chromosome 9p23
ii. a PTPRD tyrosine phosphatase gene
iii. a bc028038 gene
iv. chromosome 3q25
v. a AADAC gene
vi. a SUCNR1 gene
wherein said deletion indicates that the subject has cancer or a predisposition thereto.

14. The method of claim 13, wherein said cancer is lung cancer.

15. The method of claim 14, wherein said lung cancer is small cell lung cancer, lung adenocarcinoma or large cell carcinoma.

16. A method of alleviating a symptom of cancer in a subject, comprising:
a. identifying a subject having an elevated copy number of a nucleic acid compared to a normal non-neoplastic copy number of said nucleic acid wherein said nucleic acid is selected from the group consisting of:
i. an ASPH gene or fragment thereof;
ii. a region of human chromosome 8q12.1-q13.11;
iii. a MGC24646 gene or fragment thereof;
iv. a region of human chromosome 12p11;
v. a LOC283343 or fragment thereof;
vi. a CGI-04 gene or fragment thereof;
vii. a DNM1L gene or fragment thereof;
viii. a PKP2 gene or fragment thereof;
ix. a region of human chromosome 22q11;
x. a CRKL gene or fragment thereof; and
xi. a PIK4CA gene or fragment thereof; and
b. administering to said mammal a compound which inhibits expression of said nucleic acid or activity of a polypeptide encoded by said nucleic acid.

17. The method of claim 16, wherein said cancer is lung cancer.

18. The method of claim 17, wherein said lung cancer is small cell lung cancer, lung adenocarcinoma or large cell carcinoma.

19. The method of claim 16, wherein said compound inhibits β-hydroxylase activity.
